SEQUENCE LISTING (1 sequence)

SEQ ID NO: 1 - protein, 25 aa, Artificial Sequence
  synthetic zinger finger motif
CXXXXCXXXXXXXXXXXXHXXXXXH

(19) United States
(12) Patent Application Publication
(10) Pub. No.: US 2006/0166206 A1
(43) Pub. Date: Jul. 27, 2006

(54) Methods and compositions for analysis of regulatory sequences

(75) Inventors: Fyodor Urnov, Pt. Richmond, CA (US); Eric Rhodes, Pleasanton, CA (US)

Correspondence Address: ROBINS & PASTERNAK, 1731 EMBARCADERO ROAD, SUITE 230, PALO ALTO, CA, 94303, US

(73) Assignee: Sangamo Biosciences, Inc.

(21) Appl. No.: 10/533,847

(22) PCT Filed: Nov. 17, 2003
(86) PCT No.: PCT/US03/37044

§ 371 Date: Nov. 30, 2005

Related U.S. Application Data
(60) Provisional application No. 60/426,934, filed on Nov. 15, 2002.

(51) Int. Cl. C12Q 1/68; C12M 1/34; B05D 3/02

(52) U.S. Cl. 435/6; 435/287.2; 427/2.11

(57) ABSTRACT

Methods for constructing arrays of regulatory sequences, and the arrays so obtained, are provided. Regulatory sequences for use on the arrays are isolated based on their accessibility in cellular chromatin. A number of methods for using the arrays are disclosed, including regulatory DNA profiling, epigenome profiling, toxicological profiling and identification of in vivo binding sites of DNA binding proteins in complex genomes.

### CROSS-REFERENCE TO RELATED APPLICATIONS

This application claims the benefit of U.S. Provisional application no. 60/426,934, filed Nov. 15, 2002, which application is hereby incorporated by reference its entirety herein.

### TECHNICAL FIELD

The present disclosure is in the field of bioinformatics, gene regulation, gene regulatory sequences, gene regulatory proteins, methods of characterizing cells according to their spectra of regulatory DNA sequences, and microarray technology.

### BACKGROUND

Through a concerted worldwide effort, significant progress has been made in determining the number and location of all human genes. Current estimates suggest that there are approximately 35,000 genes in the human genome. However, in order for this knowledge about human genes to be truly useful for biological research and biomedical applications, both the location and activity of regulatory sites in the DNA that control expression of these genes must be determined. To date, relatively little progress has been made in determining the sequence and/or location of this “regulatory DNA”—the regions of the genome that are responsible for controlling gene expression. Current efforts aimed at identifying human regulatory DNA are limited to informatics approaches, for example algorithms that attempt to discern regulatory DNA from so-called “junk” DNA using cross-species comparisons as a basis for assessment. These bioinformatic methods have yielded very limited information and have not allowed for accurate and complete identification of all regulatory DNA. Other methods for localization of regulatory sequences, such as analysis of nuclease hyper sensitive sites in cellular chromatin, destrov regulatory DNA in the process of identifying it, thereby precluding the isolation and sequence determination of these regulatory sequences.

Similarly, for regulatory sequences that have been identified, there are no methods for determining whether a set of regulatory sequences is active or inactive in a particular cell or tissue type. Thus, there remains a need for compositions and methods for determining the position and/or sequence and/or activity of nucleotide sequences in the human genome that perform transcriptional regulatory functions.

Moreover, transcriptional regulatory networks in the human genome are mapped at present on a gene-by-gene basis, and no massively parallel mapping strategy exists. Attempts have been made to use genome-wide expression profiling for this purpose, but even studies conducted on the relatively simple yeast genome have demonstrated that using this approach by itself reveals transcriptional phenotype, not the underlying transcriptional program. Giaver et al. (2002) Nature 418:387-391; Birrell et al. (2002) Proc. Nat'l Acad Sci USA 99:8778-8783; Kozlova et al. (2000) Trends Endocrinol Metab 11:276-280; Nal et al. (2001) Bioessays 23:473476; Pilpel et al.

Accordingly, there is a need for methods and compositions for integrating data obtained from the following studies: comparison of a cells' transcriptional profile under normal and “diseased” conditions; computational analysis of regulatory DNA of genes that become deregulated during disease; and/or experimental genome-wide analysis of transcription factor binding in vivo.

### SUMMARY

Described herein are methods for the use of libraries of regulatory sequences obtained based on accessibility of nucleotide sequences in cellular chromatin. In particular, sequences obtained from such libraries are placed on one or more nucleic acid arrays (e.g., a microarray). Such arrays of regulatory sequences can be used for a number of purposes including, for example, characterizing the distribution of binding sites in a cellular genome for a given regulatory molecule, determination of the nature, location and sequence of active regulatory sequences in a cellular genorne, determination of whether chromatin modification (e.g., covalent histone modifications such as methylation, acetylation and/or phosphorylation) has occurred at one or more regulatory sequences in a cellular genome, determination of the effects of compounds (e.g., toxins, organic molecules) on the preceding three processes, determination of the presence of a single-nucleotide polymorphisms (SNPs) or haplotypes in a regulatory sequence in a cell, and identification of templates for microRNAs.

The methods generally involve obtaining a collection of accessible sequences, constructing an array (e.g., microarray) comprising the accessible sequences and using one or more of the arrays for hybridization to a collection of polynucleotide sequences. Use of these microarrays (also referred to as “regDNA chips”) allows any research group to rapidly determine how regulatory DNA sites are used in any cell or tissue.

In one aspect, a method for making an array is provided, the method comprising: (a) isolating a plurality of cellular polynucleotide sequences, whereby the sequences are isolated based on their accessibility in cellular chromatin; and (b) attaching each of the isolated sequences to an address on a solid support.

In another aspect, provided herein is an array comprising a plurality of accessible polynucleotide sequences, wherein: (a) the sequences are isolated based on their accessibility in cellular chromatin; and (b) each accessible sequence is located at a distinct address on a solid support. In certain embodiments, the accessible sequences are isolated from a plurality of different cell types from an organism.

In certain additional embodiments, the accessible sequences are isolated from a single cell or tissue type from an organism. In further embodiments, the accessible sequences may be isolated, for example, by (a) isolating a first plurality of cellular polynucleotide sequences, whereby the sequences are isolated based on their accessibility in cellular chromatin from a first cell; (b) isolating a second plurality of cellular polynucleotide sequences, whereby the sequences are isolated based on their accessibility in cellular chromatin from a second cell; (c) obtaining sequences that are unique to either the first or second plurality of cellular polynucleotide sequences; and (d) attaching each of the isolated sequences obtained in step (c) to an address on a solid support.

In another aspect, provided herein is a method of identifying a target sequence bound by a DNA-binding protein, the method comprising the steps of: (a) contacting at least one DNA-binding protein with one or more of the arrays described herein, under conditions such that the protein binds to accessible sequences comprising a target sequence bound by the protein; (b) removing unbound proteins; and (c) identifying the accessible sequences bound by the protein, thereby identifying target sequences for the protein. Optionally, the protein can be labeled with a detectable label.

In yet another aspect, provided herein is a method of identifying a transcription factor, the method comprising the steps of: (a) preparing a preparation of proteins from a cell; (b) contacting the isolated proteins with one or more of the arrays described herein, under conditions such that transcription factors in the protein preparation bind to accessible sequences comprising a target sequence bound by a transcription factor; (c) removing unbound proteins; and (d) identifying the proteins bound to the array. Optionally, the protein can be labeled with a detectable label.

In a still further aspect, provided herein is a method for obtaining a regulatory profile of accessible sequences in a cell, the method comprising: (a) isolating a plurality of polynucleotide sequences from the cell, whereby the sequences are isolated based on their accessibility in cellular chromatin; (b) optionally amplifying the sequences obtained in step (a); (c) optionally labeling the sequences of step (a) or (b); (d) contacting the sequences of step (a), (b) or (c) with one or more of the arrays described herein; and (e) identifying the accessible sequences bound on the array, thereby identifying sequences that are accessible in the cell.

In yet another aspect, provided herein is a method for identifying functional binding sites for a DNA-binding protein in a cell, the method comprising: (a) subjecting a cell to conditions under which DNA-binding proteins are crosslinked to their binding sites in cellular chromatin; (b) shearing the crosslinked cellular chromatin of step (a); (c) immunoprecipitating the sheared crosslinked chromatin of step (b) with an antibody which recognizes the DNA-binding protein; (d) reversing the crosslinks in the immunoprecipitate of step (c); (e) purifying the DNA from the immunoprecipitated material of step (d); (f) optionally amplifying the DNA obtained in step (e); (g) optionally labeling the DNA of step (e) or (f); (h) contacting the DNA from step (e), (f) or (g) with one or more of the arrays described herein; and (i) identifying the accessible sequences bound on the array, thereby identifying functional binding sites for the DNA-binding protein in the cell.

In a still further aspect, provided herein is a method of identifying a sequence in cellular chromatin, wherein the clromatin is covalently modified, the method comprising: (a) providing a sample of cellular chromatin; (b) optionally subjecting the chromatin of step (a) to conditions under which DNA-binding proteins are crosslinked to their binding sites in cellular chromatin; (c) shearing the cellular chromatin of step (a) or (b); (d) imnmunoprecipitating the sheared chromatin of step (c) with an antibody which recognizes a covalent chromatin modification; (e) purifying the DNA from the immunoprecipitated material of step (d); (f) optionally amplifying the DNA obtained in step (e); (g) optionally labeling the DNA of step (e) or (f); (h) contacting the DNA from step (e), (f) or (g) with one or more of the arrays described herein; and (i) identifying the accessible sequences bound on the array, thereby identifying sequences in cellular chromatin wherein the chromatin is covalently modified. In any of these methods, the cellular chromatin may be, for example, in an isolated nucleus or collection of nuclei, or in a cell.

In yet another aspect, provided herein is a method for characterizing the effects of a molecule on a cell, the method comprising: (a) contacting the cell with the molecule; (b) isolating a first plurality of polynucleotide sequences from the cell of step (a), whereby the sequences are isolated based on their accessibility in cellular chromatin; (c) optionally amplifying the sequences obtained in step (b); (d) optionally labeling the sequences of step (b) or (c); (e) contacting the sequences of step (b), (c) or (d) with one or more of the arrays described herein; and (f) identifying the accessible sequences bound on the array, thereby identifying sequences that are accessible in the cell. In certain embodiments, the method further comprises the steps of (g) providing cells that have not been contacted with the molecule; (h) isolating a second plurality of polynucleotide sequences from the cell of step (g), whereby the sequences are isolated based on their accessibility in cellular chromatin; (i) optionally amplifying the sequences obtained in step (h); (j) obtaining sequences that are unique to either the first or second plurality of polynucleotide sequences; (c) optionally amplifying the sequences obtained in step (j); (l) optionally labeling the sequences of step (i) or (j); (m) contacting the sequences of step (j), (k) or (l) with one or more of the arrays described herein; and (n) identifying the accessible sequences bound on the array, thereby identifying differences in accessible sequences bet veen cells that have and have not been contacted with the molecule.

In a still further aspect, provided herein is a method of identifying single nucleotide polymorphisms (SNPs) in regulatory sequences of an individual, the method comprising the steps of: (a) preparing a library of regulatory DNA sequences from chromatin isolated from cells from the individual; (b) optionally labeling the sequences of step (a); (c) hybridizing the sequences of step (a) or (b) to an array described herein, under stringent hybridization conditions, wherein the regulatory DNA sequences of the library hybridize to complementary accessible sequences on the array; (d) removing regulatory DNA sequences of the library that are not bound to accessible sequences on the array; and (e) identifying accessible sequences on the array that are not hybridized to regulatory DNA sequences of the library, wherein the unbound accessible sequences on the array suggest the presence of a SNP in regulatory sequences of the individual corresponding to the unbound accessible sequence.

In any of the methods described herein, the DNA-binding protein may be, for example, a transcription factor, a hormone receptor (e.g., estrogen receptor), a replication factor or a recombination factor.

In yet another aspect, provided herein is a method-for characterizing the effects of a stimulus on a cell, the method comprising: (a) subjecting the cell to the stimulus; (b) isolating a first plurality of polynucleotide sequences from the cell of step (a), whereby the sequences are isolated based on their accessibility in cellular chromatin; (c) optionally amplifying the sequences obtained in step (b); (d) optionally labeling the sequences of step (b) or (c); (e) contacting the sequences of step (b), (c) or (d) with one or more of the arrays described herein; and (f) identifying the accessible sequences bound on the array, thereby identifying sequences that are accessible in the cell. In certain embodiments, the method further comprises the steps of: (g) providing cells that have not been subjected to the stimulus; (h) isolating a second plurality of polynucleotide sequences from the cell of step (g), whereby the sequences are isolated based on their accessibility in cellular chromatin; (i) optionally amplifying the sequences obtained in step (h); (j) obtaining sequences that are unique to either the first or second plurality of polynucleotide sequences; (k) optionally amplifying the sequences obtained in step (j); (l) optionally labeling the sequences of step (j) or (k); (m) contacting the sequences of step (l), (k) or (l) with one or more of the arrays described herein; and (n) identifying the accessible sequences bound on the array, thereby identifying differences in accessible sequences between cells that have and have not been subjected to the stimulus. The stimulus may be, for example, disease state, infection, exposure to one or more drugs, stress, exposure to toxins, and combinations thereof.

### BRIEF DESCRIPTION OF THE DRAWINGS

FIG. 1 is a schematic depicting an exemplary transcriptional regulatory circuit.

FIG. 2, panels A-D, are blots depicting the location of DNAseI hypersensitive sites in vivo using clones isolated from a library of regulatory DNAs as probes. In each panel, the left lane is a control (no DNase); the middle lanes contain DNA from nuclei treated with DNAse I (increasing concentrations of DNasel indicated by the height of the wedge), and the right lane (“M”) contains a marker. The location of the hypersensitive site is indicated by a triple line; the location of the regulatory DNA clone, determined by comparison of the marker lane (labeled “M”) with additional molecular weight markers (not shown) is indicated by the horizontal arrowhead. In Panel A, the horizontal arrowhead marks the clone location at the transcription start site of the gene HSPC142 on chromosome 19. The horizontal arrowhead in Panel B depicts the clone location two klb upstream of the transcription start site of PP5395 on chromosome 10. In Panel C, the horizontal arrowhead marks the clone location sixteen kb upstream of the transcription start site of UPK3 on chromosome 22 and in Panel D, the clone is located twenty five kb downstream of the transcription start site of SARTI.

Vertical arrows in panels A and B represent portions of the transcribed region of gene; with the tail of the arrow corresponding to the transcription startsite.

FIG. 3, Panels A and B, are pie graphs depicting regulatory DNA library clone distribution (Panel A) and distribution of DNA in the genome (Panel B). Panel A depicts the location of 405 clones from a HEK 293 regulatory DNA library. Panel B depicts the expected distribution if the library contained randomly isolated 500 bp fragments from the genome.

FIG. 4 is a graph depicting mouse-human evolutionary conservation score using a nonpromoter clone from the regulatory DNA library (location on the genome indicated by the black bar at top center). The chromosomal sequence depicted includes a stretch of human chromosome 22 containing the transcription start site of the OLIG2 gene. The grayscale graph shows mouse-human sequence conservation across this region (the height of the peak corresponds to the degree of conservation). The core promoter is located at the peak on the right indicated by the arrow 1 beneath the graph. A small peak of mouse-human conservation (indicated by the number 2 beneath the graph) precisely coincides with the location of the clone from the regulatory DNA library (black bar above the graph in center).

FIG. 5 is a schematic flowchart depicting steps used in constructing an array to map intergenic yeast regions. The first three steps are essentially chromatin irniunoprecipitation (ChIP). Unlike humans, regulatory regions in yeast are intergenic. Accordingly, in yeast, the products of chromatin immunoprecipitation can be directly assessed using microarrays of yeast intergenic regions.

FIG. 6 is a flowchart depicting various steps used to assess regulatory DNA.

### DETAILED DESCRIPTION

The ability to isolate and identify human regulatory DNA on a genome-wide scale is a unique capability. The construction of microarrays comprising a plurality of regulatory sequences, isolated by virtue of their accessibility in cellular chromatin, allows many types of analysis of cellular regulatory mechanisms, as described herein.

The practice of conventional techniques in molecular biology, biochemistry, cliromatin structure and analysis, computational chemistry, cell culture, recombinant DNA, bioinfornatics, genomics and related fields are well-known to those of skill in the art and are discussed, for example, in the following literature references: Sambrook et al. MOLECULAR CLONING: A LABORATORY MANUAL, Second edition, Cold Spring Harbor Laboratory Press, 1989 and Third edition, 2001; Ausubel et al., CURRENT PROTOCOLS IN MOLECULAR BIOLOGY, John Wiley & Sons, New York, 1987 and periodic updates; the series METHODS IN ENZYMOLOGY, Academic Press, San Diego; Wolffe, CHROMATIN STRUCTURE AND FUNCTION, Third edition, Academic Press, San Diego, 1998; METHODS IN ENZYMOLOGY, Vol. 304, “Chromatin” (P. M. Wassarman and A. P. Wolffe, eds.), Academic Press, San Diego, 1999; and METHODS IN MOLECULAR BIOLOGY, Vol. 119, “Chromatin Protocols” (P. B. Becker, ed.) Humana Press, Totowa, 1999, all of which are incorporated by reference in their entireties.

I. Definitions

The terms “nucleic acid,” “polynucleotide,” and “oligonucleotide” are used interchangeably and refer to a deoxyribonucleotide or ribonucleotide polymer in either single- or double-stranded form. For the purposes of the present disclosure, these terms are not to be construed as limiting with respect to the length of a polymer. The terms can encompass known analogues of natural nucleotides, as well as nucleotides that are modified in the base, sugar and/or phosphate moieties. In general, an analogue of a particular nucleotide has the same base-pairing specificity; ie., an analogue of A will base-pair with T. The terms also encompasses nucleic acids containing modified backbone residues or linkages, which are synthetic, naturally occurring, and non-naturally occurring, which have similar binding properties as the reference nucleic acid, and which are metabolized in a manner similar to the reference nucleotides. Examples of such analogs include, without limitation, phosphorothioates, phosphoramidates, methyl phosphonates, chiral-methyl phosphonates, 2-O-methyl ribonucleotides, peptide-nucleic acids (PNAs).

Unless otherwise indicated, a particular nucleic acid sequence also implicitly encompasses conservatively modified variants thereof (e.g., degenerate codon substitutions) and complementary sequences, as well as the sequence explicitly indicated. Nucleic acids include, for example, genes, cDNAs, and mRNAs. Polynucleotide sequences are displayed herein in the conventional 5′-3′ orientation.

The terms “polypeptide,” “peptide” and “protein” are used interchangeably herein to refer to a polymer of amino acid residues. The terms apply to amino acid polymers in which one or more amino acid residue is an analog or mimetic of a corresponding naturally occurring amino acid, as well as to naturally occurring amino acid polymers. Polypeptides can be modified, e.g., by the addition of carbohydrate residues to form glycoproteins. The terms “polypeptide,” “peptide” and “protein” include glycoproteins, as well as non-glycoproteins. The polypeptide sequences are displayed herein in the conventional N-terminal to C-terminal orientation.

Binding refers to an interaction between two molecules; e.g., between two proteins, between a protein and a small molecule (molecular weight <10 kD) ligand, between a protein and a nucleic acid or between two single-stranded nucleic acids to form a nucleic acid duplex or “hybrid.” Binding can be covalent or non-covalent and can be specific or non-specific. Protein-nucleic binding and nucleic acid-nucleic acid binding is often sequence-specific, but is not necessarily so. Methods for determining sequence-specificity of binding interactions are known in the art.

Nucleotide sequence-specific binding between two single-stranded polynucleotides, mediated by base-pairing, to form a double-stranded polynucleotide, is known as “aimealing,” “hybridization” or “renaturation.” One of the two single-stranded polynucleotides is sometimes referred to as a “hybridization probe” and the other a “target” nucleic acid. A probe nucleic acid is often labeled, by methods known in the art. In this way duplex polynucleotides formed by hybridization can be detected.

Conditions for hybridization are well-known to those of skill in the art. Hybridization stringency refers to the degree to which hybridization conditions disfavor the formation of hybrids containing mismatched nucleotides, with higher stringency correlated with a lower tolerance for mismatched hybrids. Factors that affect the stringency of hybridization are well-known to those of skill in the art and include, but are not limited to, temperature, pH, ionic strength, and concentration of organic solvents such as, for example, formamide and dimethylsulfoxide. As is known to those of skill in the art, hybridization stringency is increased by higher temperatures, lower ionic strength and lower solvent concentrations.

Stringency of hybridization can also be modulated by using certain nucleotide analogues or pendant groups in one and/or the other of the hybridization probe or target nucleic acid. See, for example, U.S. Pat. Nos. 5,801,155; 6,127,121; 6,312,894; 6,485,906; and 6,492,346; and Liu et al. (2003) Science 302:868-871.

With respect to stringency conditions for hybridization, it is well known in the art that numerous equivalent conditions can be employed to establish a particular stringency by varying, for example, the following factors: the length and nature of probe and target sequences, base composition of the various sequences,. concentrations of salts and other hybridization solution components, the presence or absence of blocking agents in the hybridization solutions (e.g., dextran sulfate, and polyethylene glycol), hybridization reaction temperature and time parameters, as well as, varying wash conditions. The selection of a particular set of hybridization conditions is accomplished following standard methods in the art. See, for example, Sambrook, et al., Molecular Cloning: A Laboratory Manual, Second Edition, (1989) Cold Spring Harbor, N.Y.; Nucleic Acid Hybridization: A Practical Aproach, editors B. D. Hames and S.J. Higgins, (1985) Oxford; Washington, D.C.; IRL Press.

A “binding protein” “or binding domain” is a protein or polypeptide that is able to bind covalently or non-covalently to another molecule. Non-covalent binding includes, but is not limited to, ionic bonding, hydrogen bonding, Van der Vfaal's interactions, hydrophobic interactions or any combination of the aforementioned. A binding protein can bind to, for example, a DNA molecule (a DNA-binding protein), an RNA molecule (an RNA-binding protein) and/or a protein molecule (a protein-binding protein). In the case of a protein-binding protein, it can bind to itself (to form homodimers, homotrimers, etc.) and/or it can bind to one or more molecules of a different protein or proteins. A binding protein can have more than one type of binding activity. For example, zinc finger proteins have DNA-binding, RNA-binding and protein-binding activity.

The interaction between a DNA-binding protein and its target sequence can be characterized by its affinity and by its specificity. Affinity refers to the strength of the binding interaction and can be expressed quantitatively as a dissociation constant (K_{d}). Specificity refers to the degree to which a binding protein binds more strongly to one sequence (e.g., its target sequence) that to another related sequence. High-affinity binding between, for example, a DNA-binding protein and a specific DNA target sequence is characterized by a dissociation constant of 1×10⁻⁶ M or lower.

A “zinc finger binding protein” is a protein or polypeptide that binds DNA, RNA and/or protein, preferably in a sequence-specific manner, as a result of stabilization of protein structure through coordination of a zinc ion. The term zinc finger binding protein is often abbreviated as zinc finger protein or ZFP. The individual DNA binding domains are typically referred to as “fingers” A ZFP has least one finger, typically two fingers, three fingers, or six fingers. Each finger binds from two to four base pairs of DNA, typically three or four base pairs of DNA. A ZFP binds to a nucleic acid sequence called a target site or target segment. Each finger typically comprises an approximately 30 amino acid, zinc-chelating, DNA-binding subdomain. An exemplary motif characterizing one class of these proteins (C₂H₂ class) is -Cys-(X)₂₋₄-Cys-(X)₁₂-His-(X)₃₋₅-His (where X is any amino acid) (SEQ ID NO: 1). Studies have demonstrated that a single zinc finger of this class consists of an alpha helix containing the two invariant histidine residues coordinated with zinc along with the two cysteine residues of a single beta turn (see, e.g., Berg & Shi, Science 271:1081-1085 (1996)).

Zinc finger binding domains can be engineered to bind to a predetermined nucleotide sequence. Non-limiting examples of methods for engineering zinc finger proteins are design and selection.

A “designed” zinc finger protein is a protein not occurring in nature whose structure and composition result principally from rational criteria. Rational criteria for design include application of substitution rules and computerized algorithms for processing information in a database storing infonnation of existing ZFP designs and binding data, for example as described in co-owned U.S. Pat. No. 6,453,242. See also U.S. Pat. Nos. 6,140,081 and 6,534,261 and WO 98/53058; WO 98/53059; WO 98/53060; WO 02/016536 and WO 03/016496. A “selected” zinc finger protein is a protein not found in nature whose production results primarily from an empirical process such as phage display, interaction trap or hybrid selection. See e.g., U.S. Pat. No. 5,789,538; U.S. Pat. No. 5,925,523; U.S. Pat. No. 6,007,988; U.S. Pat. No. 6,013,453; U.S. Pat. No. 6,200,759; WO 95/19431; WO 96/06166; WO 98/53057; WO 98/54311; WO 00/27878; WO 01/60970 WO 01/88197 and WO 02/099084.

A “target site” or “target sequence” is a sequence that is bound by a binding protein such as, for example, a ZFP. Target sequences can be nucleotide sequences (either DNA or RNA) or amino acid sequences. A single target site typically has about four to about ten base pairs. Typically, a two-fingered ZFP recognizes a four to seven base pair target site, a three-fingered ZFP recognizes a six to ten base pair target site, and a six fingered ZFP recognizes two adjacent nine to ten base pair target sites. By way of example, a DNA target sequence for a three-finger ZFP is generally either 9 or 10 nucleotides in length, depending upon the presence and/or nature of cross-strand interactions between the ZFP and the target sequence.

Target sequences can be found in any DNA or RNA sequence, including regulatory sequences, exons, introns, or any non-coding sequence.

A “target subsite” or “subsite” is the portion of a DNA target site that is bound by a single zinc finger, excluding cross-strand interactions. Thus, in the absence of cross-strand interactions, a subsite is generally three nucleotides in length. In cases in which a cross-strand interaction occurs (e.g., a “D-able subsite,” as described for example co-owned U.S. Pat. No. 6,453,242, incorporated by reference in its entirety herein, a subsite is four nucleotides in length and overlaps with another 3- or 4-nucleotide subsite.

Chromatin is the nucleoprotein structure comprising the cellular genome. “Cellular chromatin” comprises nucleic acid, primarily DNA, and protein, including histones and non-histone chromosomal proteins. The majority of eukaryotic cellular chromatin exists in the forn of nucleosornes, wherein a nucleosome core comprises approximately 150 base pairs of DNA associated with an octamer comprising two each of histones H2A, H2B, H3 and H4; and linker DNA (of variable length depending on the organism) extends between nucleosome cores. A molecule of histone HI is generally associated with the linker DNA. For the purposes of the present disclosure, the term “chromatin” is meant to encompass all types of cellular nucleoprotein, both prokaryotic and eukaryotic. Cellular chromatin includes both chromosomal and episomal chromatin.

A “chromosome” is a chromatin complex comprising all or a portion of the genome of a cell. The genome of a cell is often characterized by its karyotype, which is the collection of all the chromosomes that comprise the genome of the cell. The genome of a cell can comprise one or more chromosomes.

An “episome” is a replicating nucleic acid, nucleoprotein complex or other structure comprising a nucleic acid that is not part of the chromosomal karyotype of a cell. Examples of episomes include plasmids and certain viral genomes.

An “accessible region” in cellular chromatin is generally one that does not have a typical nucleosomal structure. As such, an accessible region can be identified and localized by, for example, the use of chemicals and/or enzymes that probe chromatin structure. Accessible regions will, in general, have an altered reactivity to a probe, compared to bulk chromatin. An accessible region may be sensitive to the probe, compared to bulk chromatin, or it may have a pattern of sensitivity that is different from the pattern of sensitivity exhibited by bulk chromatin. Accessible regions can be identified by any method known to those of skill in the art for probing chromatin structure.

In one embodiment, an enzymatic probe of chromatin structure is used to identify an accessible region. In a preferred embodiment, the enzymatic probe is DNase I (pancreatic deoxyribonuclease). Regions of cellular chromatin that exhibit enhanced sensitivity to digestion by DNase I, compared to bulk chromatin (i.e., DNase-hypersensitive sites) are more likely to have a structure that is favorable to the binding of an exogenous molecule, since the nucleosomal structure of bulk chromatin is generally less conducive to binding of an exogenous molecule. Furthermore, DNase-hypersensitive regions of chromatin often contain DNA sequences involved in the regulation of gene expression. Thus, binding of an exogenous molecule to a DNase-hypersensitive chromatin region is more likely to have an effect on gene regulation.

In a separate embodiment, micrococcal nuclease ease) is used as a probe of chromatin structure to identify an accessible region. MNase preferentially digests the linker DNA present between nucleosomes, compared to bulk chromatin. It is likely that such linker DNA sequences are more apt to be bound by an exogenous molecule that are sequences present in nucleosomal DNA, which is wrapped around a histone octamer.

Additional enzymatic probes of chromatin structure include, but are not limited to, exonuclease III, S1 nuclease, mung bean nuclease, DNA methyltransferases and restriction endonucleases. In addition, the method described by van Steensel et al. (2000) Nature Biotechnology 18:424428 can be used to identify an accessible region.

Chemical probes of chromatin structure, useful in the identification of accessible regions, include, but are not limited to, hydroxyl radicals, methidiumpropyl-EDTA.Fe(II) (MPE) and crosslinkers such as psoralen. See, for example, Tullius et al. (1987) Meth. Enzymology, Vol. 155, (J. Ableson & M. Simon, eds.) Academic Press, San Diego, pp. 537-558; Cartwright et al. (1983) Proc. Natl. Acad. Sci. USA 80:3213-3217; Hertzberg et al. (1984) Biochemistry 23:3934-3945; and Wellinger et al. in Methods in Molecular Biology, Vol. 119 (P. Becker, ed.) Humana Press, Totowa, N.J., pp. 161-173.

It will clear that the aforementioned “probes of chromatin structure” are distinct from the “hybridization probes” also disclosed herein, and the differences will be clear to one of skill in the art.

A “gene,” for the purposes of the present disclosure, includes a DNA region encoding a gene product, as well as all DNA regions that regulate the production of the gene product, whether or not such regulatory sequences are adjacent to coding and/or transcribed sequences. Accordingly, a gene includes, but is not necessarily limited to, promoter sequences, terminators, translational regulatory sequences such as ribosome binding sites and internal ribosome entry sites, enhancers, silencers, insulators, boundary elements, replication origins, matrix attachment sites and locus control regions.

“Gene expression” refers to the conversion of the information, contained in a gene, into a gene product. A gene product can be the direct transcriptional product of a gene (e.g., mRNA, tRNA, rRNA, antisense RNA, ribozyme, structural RNA or any other type of RNA) or a protein produced by translation of a MnRNA. Gene products also include RNAs that are modified, by processes such as capping, polyadenylation, methylation, and editing, and proteins modified by, for example, methylation, acetylation, phosphorylation, ubiquitination, ADP-ribosylation, myristilation, and glycosylation.

“Gene activation” and “augmentation of gene expression” refer to any process that results in an increase in production of a gene product. A gene product can be either RNA (including, but not limited to, mRNA, rRNA, tRNA, and structural RNA) or protein. Accordingly, gene activation includes those processes that increase transcription of a gene and/or translation of a MRNA. Examples of gene activation processes which increase transcription include, but are not limited to, those which facilitate formation of a transcription initiation complex, those which increase transcription initiation rate, those which increase transcription elongation rate, those which increase processivity of transcription and those which relieve transcriptional repression (by, for example, blocking the binding of a transcriptional repressor). Gene activation can constitute, for example, inhibition of repression as well as stimulation of expression above an existing level. Examples of gene activation processes which increase translation include those which increase translational initiation, those which increase translational elongation and those which increase mRNA stability In general, gene activation comprises any detectable increase in the production of a gene product, preferably an increase in production of a gene product by about 2-fold, more preferably from about 2- to about 5-fold or any integer therebetween, more preferably between about 5- and about 10-fold or any integer therebetween, more preferably between about 10- and about 20-fold or any integer therebetween, still more preferably between about 20- and about 50-fold or any integer therebetween, more preferably between about 50- and about 100-fold or any integer therebetween, more preferably 100-fold or more.

“Gene repression” and “inhibition of gene expression” refer to any process that results in a decrease in production of a gene product. A gene product can be either RNA (including, but not limited to, mRNA, rRNA, tRNA, and structural RNA) or protein. Accordingly, gene repression includes those processes that decrease transcription of a gene and/or translation of a mRNA. Examples of gene repression processes which decrease transcription include, but are not limited to, those which inhibit formation of a transcription initiation complex, those which decrease transcription initiation rate, those which decrease transcription elongation rate, those which decrease processivity of transcription and those which antagonize transcriptional activation (by, for example, blocking the binding of a transcriptional activator). Gene repression can constitute, for example, prevention of activation as well as inhibition of expression below an existing level. Examples of gene repression processes that decrease translation include those that decrease translational initiation, those that decrease translational elongation and those that decrease mRNA stability. Transcriptional repression includes both reversible and irreversible inactivation of gene transcription. In general, gene repression comprises any detectable decrease in the production of a gene product, preferably a decrease in production of a gene product by about 2-fold, more preferably from about 2- to about 5-fold or any integer therebetween, more preferably between about 5- and about 10-fold or any integer therebetween, more preferably between about 10- and about 20-fold or aniy integer therebetween, still more preferably between about 20- and about 50-fold or any integer tlherebetween, more preferably between about 50- and about 100-fold or any integer therebetween, more preferably 100-fold or more. Most preferably, gene repression results in complete inhibition of gene expression, such that no gene product is detectable.

The term “modulate” refers to a change in the quantity, degree or extent of a function. For example, the modified zinc finger-nucleotide binding polypeptides disclosed herein may modulate the activity of a promoter sequence by binding to a motif within the promoter, thereby inducing, enhancing or suppressing transcription of a gene operatively linked to the promoter sequence. Altematively, modulation may include inhibition of transcription of a gene wherein the modified zinc finger-nucleotide binding polypeptide binds to the structural gene and blocks DNA dependent RNA polymerase from reading through the gene, thus inhibiting transcription of the gene. The structural gene may be a normal cellular gene or an oncogene, for example. Alternatively, modulation may include inhibition of translation of a transcript. Thus, “modulation” of gene expression includes both gene activation and gene repression.

Modulation can be assayed by determining any parameter that is indirectly or directly affected by the expression of the target gene. Such parameters include, e.g., changes in RNA or protein levels; changes in protein activity; changes in product levels; changes in downstream gene expression; changes in transcription or activity of reporter genes such as, for example, luciferase, CAT, beta-galactosidase, or GFP (see, e.g., Mistili & Spector, (1997) Nature Biotechnology) 15:961-964); changes in signal transduction; changes in phosphorylation and dephosphorylation; changes in receptor-ligand interactions; changes in concentrations of second messengers such as, for example, cGMP, cAMP, IP3, and Ca2⁺; changes in cell growth, changes in neovascularization, and/or changes in any functional effect of gene expression. Measurements can be made iin vitro, ini vivo, and/or ex vivo. Such functional effects can be measured by conventional methods, e.g., measurement of RNA or protein levels, measurement of RNA stability, and/or identification of downstream or reporter gene expression. Readout can be by way of, for example, chemiluminescence, fluorescence, colorimetric reactions, antibody binding, inducible markers, ligand binding assays; changes in intracellular second messengers such as cGMP and inositol triphosphate (IP₃); changes in intracellular calcium levels; cytokine release, and the lice.

Accordingly, the terms “modulating expression” “inhibiting expression” and “activating expression” of a gene can refer to the ability of a molecule to activate or inhibit transcription of a gene. Activation includes prevention of transcriptional inhibition (i.e., prevention of repression of gene expression) and inhibition includes prevention of transcriptional activation (i.e., prevention of gene activation).

A “functional fragment” of a protein, polypeptide or nucleic acid is a protein, polypeptide or nucleic acid whose sequence is not identical to the full-length protein, polypeptide or nucleic acid, yet retains the same function as the full-length protein, polypeptide or nucleic acid. A functional fragment can possess more, fewer, or the same number of residues as the corresponding native molecule, and/or can contain one ore more amino acid or nucleotide substitutions. Methods for determining the function of a nucleic acid (e.g., coding. function, ability to hybridize to another nucleic acid) are well-known in the art. Similarly, methods for determining protein function are well-known. For example, the DNA-binding function of a polypeptide can be determined, for example, by filter-binding, electrophoretic mobility-shift, or immunoprecipitation assays. See Ausubel et al., suppra. The ability of a protein to interact with another protein can be determined, for example, by co-immunoprecipitation, two-hybrid assays or complementation, both genetic and biochemical. See, for example, Fields et al. (1989) Nature 340:245-246; U.S. Pat. No. 5,585,245 and PCT WO 98/44350.

A “fusion molecule” is a molecule in which two or more subunit molecules are linked, preferably covalently. The subunit molecules can be the same chemical type of molecule, or can be different chemical types of molecules. Examples of the first type of fusion molecule include, but are not limited to, fusion polypeptides (for example, a fusion between a ZFP DNA-binding domain and a transcriptional activation domain) and fusion nucleic acids (for example, a nucleic acid encoding the fusion polypeptide described herein). Examples of the second type of fusion molecule include, but are not limited to, a fusion between a triplex-forming nucleic acid and a polypeptide, and a fusion between a minor groove binder and a nucleic acid.

The term “heterologous” is a relative term, which when used with reference to portions of a nucleic acid indicates that the nucleic acid comprises two or more subsequences that are not found in the same relationship to each other in nature. For instance, a nucleic acid that is recombinantly produced typically has two or more sequences from unrelated genes synthetically arranged to make a new functional nucleic acid, e.g., a promoter from one source and a coding region from another source. The two nucleic acids are thus heterologous to each other in this context. When added to a cell, the recombinant nucleic acids would also be heterologous to the endogenous genes of the cell. Thus, in a chromosome, a heterologous nucleic acid would include an non-native (non-naturally occurring) nucleic acid that has integrated into the chromosome, or a non-native (non-naturally occurring) extrachromosomal nucleic acid. In contrast, a naturally translocated piece of chromosome would not be considered heterologous in the context of this patent application, as it comprises an endogenous nucleic acid sequence that is native to the mutated cell.

Similarly, a heterologous protein indicates that the protein comprises two or more subsequences that are not found in the same relationship to each other in nature (e.g., a “fusion protein,” where the two subsequences are encoded by a single nucleic acid sequence). See, e.g., Ausubel, supra, for an introduction to recombinant techniques.

The term “recombinant when used with reference, e.g., to a cell, or nucleic acid, protein, or vector, indicates that the cell, nucleic acid, protein or vector, has been modified by the introduction of a heterologous nucleic acid or protein or the alteration of a native nucleic acid or protein, or that the cell is derived from a cell so modified. Thus, for example, recombinant cells express genes that are not found within the native (naturally occurring) form of the cell or express a second copy of a native gene that is otherwise normally or abnormally expressed, under expressed or not expressed at all.

Nucleic acid or amino acid sequences are “operably linked” (or “operatively linked”) when placed into a functional relationship with one another. For instance, a promoter or enhancer is operably linked to a coding sequence if it regulates, or contributes to the modulation of, the transcription of the coding sequence. Operably linked DNA sequences are typically contiguous, and operably linked amino acid sequences are typically contiguous and in the same reading frame. However, since enhancers generally function when separated from the promoter by up to several kilobases or more and intronic sequences may be of variable lengths, some polynucleotide elements may be operably linked but not contiguous. Similarly, certain amino acid sequences that are non-contiguous in a primary polypeptide sequence may nonetheless be operably linked due to, for example folding of a polypeptide chain.

With respect to fusion polypeptides, the terms “operatively linked” and “operably linked” can refer to the fact that each of the components performs the same function in linkage to the other component as it would if it were not so linked. For example, with respect to a fusion polypeptide in which a ZFP DNA-binding domain is fused to a transcriptional activation domain (or functional fragment thereof), the ZFP DNA-binding domain and the transcriptional activation domain (or functional fragment thereof) are in operative linkage if, in the fusion polypeptide, the ZFP DNA-binding domain portion is able to bind its target site and/or its binding site, while the transcriptional activation domain (or functional fragment thereof) is able to activate transcription.

An “expression vector” is a nucleic acid construct, generated recombinantly or synthetically, with a series of specified nucleic acid elements that permit transcription of a particular nucleic acid in a host cell, and optionally integration or replication of the expression vector in a host cell. The expression vector can be part of a plasmid, virus, or nucleic acid fragment, of viral or non-viral origin. Typically, the expression vector includes an “expression cassette,” which comprises a nucleic acid to be transcribed operably linked to a promoter. The term expression vector also encompasses naked DNA operably linked to a promoter.

“Eucaryotic cells” include, but are not limited to, fungal cells (such as yeast), plant cells, animal cells, mammalian cells and human cells.

The term “comrnon,” when used in reference to two or more polynucleotide sequences being compared, refers to polynucleotides that (i) exhibit a selected percentage of sequence identity (as defined below, typically between 80-100% sequence identity) and/or (ii) are located in similar positions, relative to a gene of interest. Likewise, the term “unique,” when used in reference to two or more polynucleotide sequences being compared, refers to polynucleotides that (i) do not exhibit a selected percentage of sequence identity as defined below, typically less than 80% sequence identity) and/or (ii) are located in one or more different positions relative to a gene of interest.

“Sequence similarity” refers to the percent similarity in base pair sequence (as determined by any suitable method) between two or more polynucleotide sequences. Two or more sequences can be anywhere from 0-100% similar, or any integer value therebetween. Furthermore, sequences are considered to exhibit “sequence identity” when they are at least about 80-85%, preferably at least about 85-90%, more preferably at least about 90-92%, more preferably at least about 93-95%, more preferably 96-98%, and most preferably at least about 98-100% sequence identity (including all integer values falling within these described ranges). These percent identities are, for example, relative to the claimed sequences, or other sequences, when the sequences obtained by the methods disclosed herein are used as the query sequence. Additionally, one of skill in the art can readily determine the proper search parameters to use for any given sequence in the programs described herein. For example, the search parameters may vary based on the size of the sequence in question. Thus, for example, in certain embodiments, the search is conducted based on the size of the isolated polynucleotide(s) corresponding to an accessible region. The isolated polynucleotide comprises X contiguous nucleotides and is compared to the sequences of approximately same length, preferably the same length. Exemplary fragment lengths include, but are not limited to, at least about 6-1000 contiguous nucleotides (or any integer therebetween), at least about 50-750 contiguous nucleotides (or any integer therebetween), about 100-300 contiguous nucleotides (or any integer therebetween), wherein such contiguous nucleotides can be derived from a larger sequence of contiguous nucleotides.

Techniques for determining nucleic acid and amino acid sequence similarity are known in the art. Typically, such techniques include determining the nucleotide sequence of, e.g., an accessible region of cellular cliromatin, and comparing these sequences to a second nucleotide sequence. Genomic sequences can also be determined and compared in this fashion. In general, “identity” refers to an exact nucleotide-to-nucleotide or amino acid-to-amino acid correspondence of two polynucleotides or polypeptide sequences, respectively. Two or more sequences (polynucleofide or amino acid) can be compared by determining their “percent identity.” The percent identity of two sequences, whether nucleic acid or amino acid sequences, is the number of exact matches between two aligned sequences divided by the length of the shorter sequences and multiplied by 100. An approximate alignment for nucleic acid sequences is provided by the local homology algorithm of Smith and Waterman, Advances in Applied Mathematics 2:482489 (1981). This algorithm can be applied to amino acid sequences by using the scoring matrix developed by Dayhoff, Atlas of Protein Sequences and Structure, M. O. Dayhoff ed., 5 suppl. 3:353-358, National Biomedical Research Foundation, Washington, D.C., USA, and normalized by Gribskov, Nucl. Acids Res. 14(6):6745-6763 (1986). An exemplary implementation of this algorithm to determine percent identity of a sequence is provided by the Genetics Computer Group (Madison, Wis.) in the “BestFit” utility application. The default parameters for this method are described in the Wisconsin Sequence Analysis Package Program Manual, Version 8 (1995) (available from Genetics Computer Group, Madison, Wis.). An additional method of establishing percent identity in the context of the present disclosure is to use the MPSRCH package of programs copyrighted by the University of Edinburgh, developed by John F. Collins and Shane S. Sturrok, and distributed by IntelliGenetics, Inc. (Mountain View, Calif.). From this suite of packages the Smith-Waterman algorithm can be employed where default parameters are used for the scoring table (for example, gap open penalty of 12, gap extension penalty of one, and a gap of six). From the data generated the “Match” value reflects “sequence identity.” Other suitable programs for calculating the percent identity or similarity between sequences are generally known in the art, for example, another alignment program is BLAST, used with default parameters. For example, BLASTN and BLASTP can be used using the following default parameters: genetic code=standard; filter=none; strand=both; cutoff=60; expect=10; Matrix=BLOSUM62; Descriptions=50 sequences; sort by=HIGH SCORE; Databases=non-redundant, GenBank+EMBL+DDBJ+PDB+GenBank CDS translations+Swiss protein+Spupdate+PIR. Details of these programs can be found at the following intemet address: http://www.ncbi.nlm.gov/cgi-bin/BLAST. When claiming sequences relative to sequences described herein, the range of desired degrees of sequence identity is approximately 80% to 100% and any integer value therebetween. Typically the percent identities between the disclosed sequences and the claimed sequences are at least 70-75%, preferably 80-82%, more preferably 85-90%, even more preferably 92%, still more preferably 95%, and most preferably 98% sequence identity to the reference sequence.

An “exogenous molecule” is a molecule that is not normally present in a cell, but can be introduced into a cell by one or more genetic, biochemical or other methods. Normal presence in the cell is determined with respect to the particular developmental stage and environmiental conditions of the cell. Thus, for example, a molecule that is present only during embryonic development of muscle is an exogenous molecule with respect to an adult muscle cell. Similarly, a molecule induced by heat shock is an exogenous molecule with respect to a non-heat-shocked cell. An exogenous molecule can comprise, for example, a functioning version of a malfunctioning endogenous molecule or a malfunctioning version of a normally-functioning endogenous molecule. Thus, the term “exogenous regulatory molecule” refers to a molecule that can modulate gene expression in a target cell but which is not encoded by the cellular genome of the target cell.

An exogenous molecule can be, among other things, a small molecule (ie., molecular weight less than 10 kD), such as is generated by a combinatorial chemistry process, or a macromolecule such as a protein, nucleic acid, carbohydrate, lipid, glycoprotein, lipoprotien, polysacchafide, any modified derivative of the above molecules, or any complex comprising one or more of the above molecules. Nucleic acids include DNA and RNA, can be single- or double-stranded; can be linear, branched or circular; and can be of any length. Nucleic acids include those capable of forming duplexes, as well as triplex-forming nucleic acids. See, for example, U.S. Pat. Nos. 5,176,996 and 5,422,251. Proteins include, but are not limited to, DNA-binding proteins, transcription factors, chromatin remodeling factors, methylated DNA binding proteins, polymerases, methylases, demethylases, acetylases, deacetylases, kinases, phosphatases, integrases, recombinases, ligases, topoisomerases, gyrases and helicases.

An exogenous molecule can be the same type of molecule as an endogenous molecule, e.g., protein or nucleic acid (i.e., an exogenous gene), providing it has a sequence that is different from an endogenous molecule. For example, an exogenous nucleic acid can comprise an infecting viral genome, a plasmid or episome introduced into a cell, or a chromosome that is not normally present in the cell. Methods for the introduction of exogenous molecules into cells are known to those of skill in the art and include, but are not limited to, lipid-mediated transfer (i.e., liposomes, including neutral and cationic lipids), electroporation, direct injection, cell fusion, particle bombardment, calcium phosphate co-precipitation, DEAE-dextran-mediated transfer and viral vector-mediated transfer.

By contrast, an “endogenous molecule” is one that is normally present in a particular cell at a particular developmental stage under particular environmental conditions. For example, an endogenous nucleic acid can comprise a chromosome, the genome of a mitochondrion, chloroplast or other organelle, or a naturally-occurring episomal nucleic acid.

Additional endogenous molecules can include proteins, for example, transcription factors and components of chromatin remodeling complexes.

Thus, an “endogenous cellular gene” refers to a gene that is native to a cell, which is in its normal genomic and chromatin context, and which is not heterologous to the cell. Such cellular genes include, e.g., animal genes, plant genes, bacterial genes, protozoal genes, fungal genes, mitrochondrial genes, and chloroplastic genes.

An “endogenous gene” refers to a microbial or viral gene that is part of a naturally occurring microbial or viral genome in a microbially or virally infected cell. The microbial or viral genome can be extrachromosomal or integrated into the host chromosome. This term also encompasses endogenous cellular genes, as described above.

The term “naturally-occurring” is used to describe an object that can be found in nature, as distinct from being artificially produced by a human. Similarly, the term “non-naturally-occurring” refers to an object or composition not found in nature.

II. General Overview

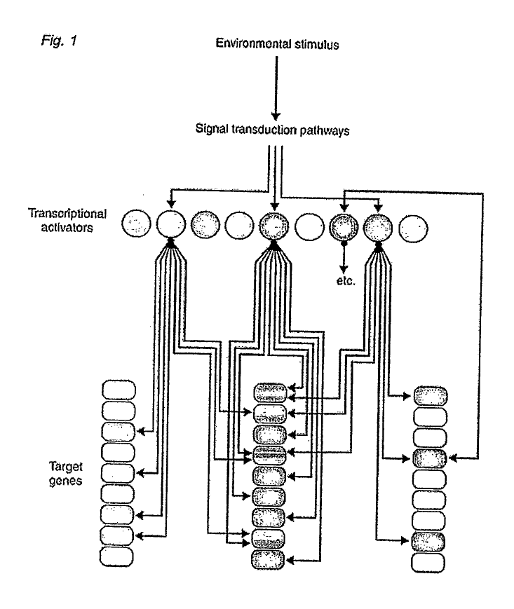

Transcription control pathways underlie nearly every major transition in cell, tissue, and organ behavior that occurs during human development and disease. As shown in FIG. 1, transcriptional pathways contain three components: (i) an environmental or developmental stimulus, such as a rise in hormone concentration, or a particular form of cell-cell interaction; (ii) a set of transcription factors that respond to the stimulus (directly or indirectly, e.g., via a signaling cascade); (iii) a set of downstream target genes that these transcription factors control by engaging DNA sequences that lie within regulatory DNA elements of these genes, such as promoters and enhancers. Disruption of normal transcription pathways often results in disease or pathology, for example aberrant function of transcription factors at these regulatory DNA stretches directly causes a considerable proportion of human disease, including, but not limited to such diseases as cancer (e.g., breast, ovarian, uterine, prostate, leukemia, lymphoma, etc.); osteoporosis; and asthma.

The first and second components of transcriptional networks have been well studied. Indeed, to date over 2,000 different transcription factors have been identified. In addition, pharmaceutical compounds that specifically affect function of these transcription factors are widely used in clinical practice as therapies, and a great many more are currently undergoing clinical trials.

However, little has been learned about the third component of transcriptional regulatory networks, target genes and their regulatory regions. Thus, although the stimulus and transcription factors associated with many transcriptional networks (e.g., hormone response systems such as estrogen, glucocorticoid, vitamin D, thyroid hormone, progesterone, testosterone, and retionic acid; cell cycle systems involving transcription factors such as myc, fos, jun, pRb, p53, E2F, etc; and inflammation pathways such as those involving NF-κB) are known, very little is known about the downstream targets (e.g., genes). For example, the direct targets of the estrogen receptor, or of niyc, are poorly defined. This lack of knowledge represents a major obstacle to malking progress in developing novel, more effective small molecule compounds that correct the dysfunction of such networks in disease. Table 1 shows a general overview of the some of the issues addressed, and technical barriers overcome, by the present disclosure.

By providing a collection of regulatory sequences active in a cell under a given set of conditions, the present disclosure allows those regulatory sequences to be associated with the gene(s) they regulate, thereby providing new information on the identity of genes whose transcription is regulated, e.g., by external stimuli, a particular transcription factor, etc.

TABLE 1

| | Technical | Current | | |
|---|---|---|---|---|
| Issues | Targets | Practice | Technical Barriers | Solution/Approach |
| Mapping regulatory | Experimental | <20% | Regulatory DNA cannot | Massively parallel |
| DNA elements in the | identification of | regulatory | be comprehensively | isolation and cloning |
| human genome | all regulatory | DNA is | identified by computation. | procedure for all active |
| | DNA elements in | identified | No high-throughput | regulatory DNA |
| | the human | | experimental approach | |
| | genome. | | exists. | |
| Comprehensive | Single-step | <5% after | No high-throughput | Use regulatory DNA |
| identification of direct | identification of | laborious | method available | microarray for high |
| genomic targets for | in vivo binding | approach | | throughput analysis of |
| transcription factors | sites for any | | | transcription factor |
| | factor | | | binding |
| Comprehensive | Identify specific | Laborious | No existing means of | Use regulatory DNA |
| mapping of | transcriptional | gene-by- | uncovering shared | microarray for massively |
| transcriptional | regulatory | gene | regulatory pathways. | parallel mapping of all |
| networks and their | pathway driving | analysis | | regulatory DNA relevant |
| misregulation in | disease | | | to a particular circuit. |
| disease | progression | | | |
| Identification of the | Identify global | Genome- | No information on what | Use of regulatory DNA |
| genome's functional | transcription | wide | controls gene expression | microarray to identify in a |
| state in a given | regulatory circuit | expression | | single step the subset of |
| cell/tissue type. | defining cell | profiling. | | regulatory DNA active in |
| | phenotype | | | a given cell type. |

III. Isolation of Regulatory Sequences

A. General

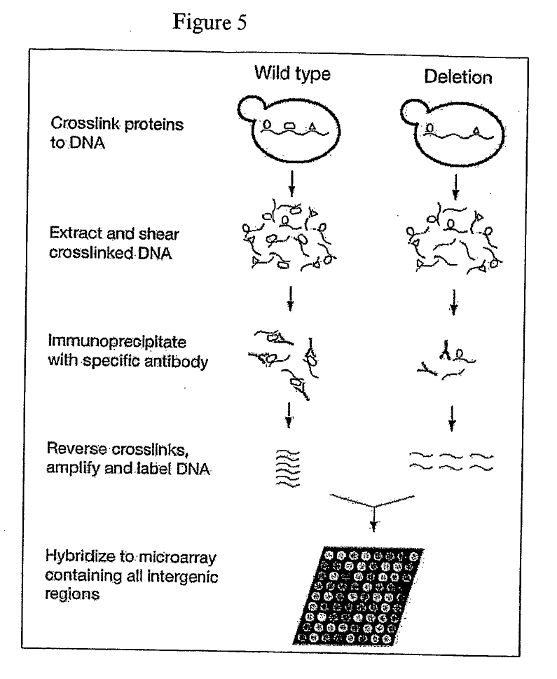

Regulatory sequences are estimated to occupy between 1 and 10% of the human genome. Approximately 80% of these regulatory DNA stretches have not been identified, largely because, unlike organisms like yeast, not all human regulatory regions occur via core promoter elements adjacent to genes (i.e., in intergenic regions of the genome). See, Wyrick et al. (2002) Curr. Opin Genet Dev 12:130-136; Nal et al. (2001) Bioessays 23:473-476. In yeast, regulatory sequences can be readily analyzed by direct mapping (Ren et al. (2000) Science 290:2306) and/or by examination of intergenic regions in response to a stimulus (Pilpel et al. (2001) Nat Genet 29:153-159. See, also, FIG. 5. However, such methods are currently inapplicable to the human genome, because for any given human gene, regulatory sequences are more complex, since they include not only core promoters but, in addition, may also include distal promoter(s), enhancer(s), insulator(s), silencer(s), boundary element(s), locus control region(s), polyA addition sites, sites involved in control of replication (e.g., replication origins), centromeres, telomeres, transcription termination sites, sites regulating chromosome structure, matrix/scaffold attachment region(s), etc. See, for example, Wingender et al. (1997) Nucleic Acids Res. 25:265-268. Moreover, these regulatory regions are typically relatively short (˜200 bp) and are dispersed widely through the genome. For instance, known regulatory elements that control β-globin gene expression include five separate approximately 200 bp sequences spread over 15,000 bp of the genome and 30,000 bp upstream of the gene's start site. In view of the complexity of human regulatory sequences, computational analysis of genome sequences in humans has not been able to identify regulatory DNA in the human genome. Pennacchio et al. (2001) Nat Rev Genet 2:100-109; Galas etal. (2001) Science 291:1257-1260.

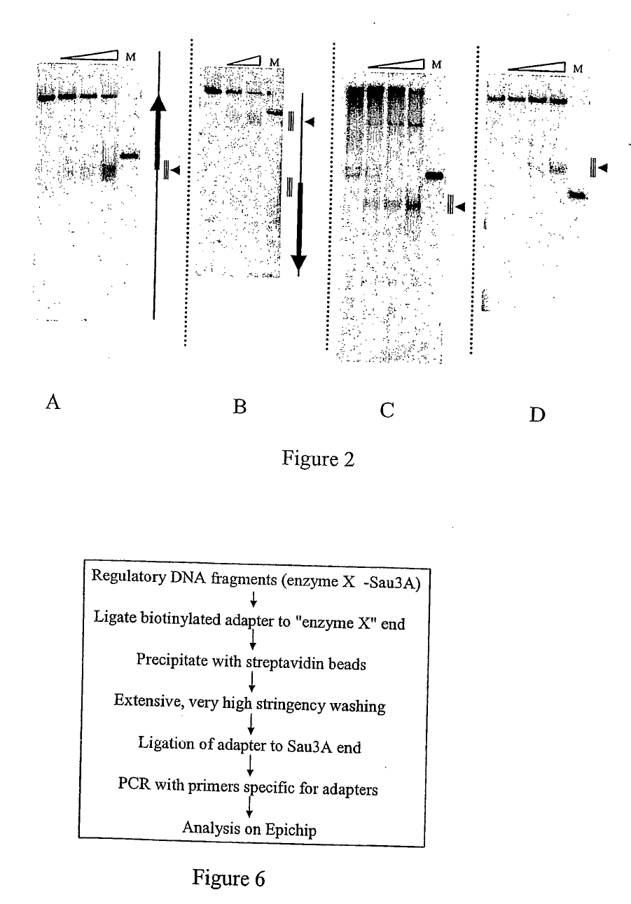

The failure of computational methods to identify regulatory regions in the human genome indicates that a different, likely experimental, solution will be required. For example, sensitivity of accessible regions to nucleases such as DNAseI is a known property of eukaryotic regulatory DNA stretches. See, e.g., Elgin et al. (1988) J. Biol. Chem. 263:19259-19262; Grosset al. (1988) Ann Rev Biochen′ 57:159-157. The accessibility of DNA in chromatin refers to any property that distinguishes a particular region of DNA, in cellular chromatin, from bulk cellular DNA. See, for example, Wolffe “Chromatin: Structure and Function” 3rd Ed., Academic Press, San Diego, 1998 for a description of cellular chromatin. For example, an accessible sequence (or accessible region) can be one that is not packaged into nucleosomes, or can comprise DNA present in nucleosomal structures that are different from that of bulk nucleosomal DNA (e.g., nucleosomes comprising modified histones). An accessible region includes, but is not limited to, a site in chromatin at which an enzymatic (e.g., DNAseI) or chemical probe reacts, under conditions in which the probe does not react with similar sites in bulk chromatin. Such regions of chromatin can include, for example, a functional group of a nucleotide, in which case probe reaction can generate a modified nucleotide, or a phosphodiester bond between two nucleotides, in which case probe reaction can generate polynucleotide fragments or chromatin fragments. Depending on the cell type or individual, chromatin includes various regions that are more or less accessible. Accessible regions in cellular chromatin may also be “remodeled,” for example, following binding of non-histone proteins to chromatin that may cause localized changes in chromatin structure and confer a dramatic (often at least an order of magnitude), but highly localized (approximately 200 bp), increase in accessibility of the regulatory DNA region to nucleases, such as DNAse I, or restriction enzymes. Increased accessibility to nucleases is commonly detected using the DNAse I hypersensitivity assay, which identifies the genomic position of these regions, lnown as “DNAse I hypersensitive sites.” See, also, FIG. 2. Although regulatory sequences may be identified on the basis of their accessibility in cellular chromatin, traditional methods of identifying regulatory sequences based on such accessibility (e.g., a locus-by-locus analysis involving DNase treatment, Southern-blotting and indirect end-labeling) is exceedingly labor intensive—mapping all regulatory sequences in the genome of a cell would take approximately 2,400 person/years using these approaches. Moreover, these methods destroy the regulatory sequences in the process of identifying them so that, although a rough location of the regulatory sequence is obtained, its nucleotide sequence is not.

Unlike the aforementioned traditional mapping methods, the methods described herein allow for both isolation and characterization of regulatory regions, and allow the isolation of a plurality of regulatory sequences in a single experiment, without requiring knowledge of the functional properties of the sequences. In other words, regulatory regions are notjust mapped, they are actually isolated (e.g., cloned) and, optionally, sequenced or otherwise characterized. See, also, International Publication WO 01/83732, incorporated herein by reference in its entirety. Once cloned, a collection of isolated regulatory sequences can be attached to an array and used in additional methods of assessing cellular regulatory processes.

B. Obtaining Marked or Modified Fragments

1. Generally

Certain methods for identifying accessible regions involve the use of an enzymatic probe that modifies DNA in chromatin. Modified regions, which comprise accessible sequences, are then identified and can be isolated. Such methods generally comprise the treatment of cellular chromatin with a chenmical and/or enzymatic probe wherein the probe reacts with (e.g., binds to, covalently modifies or cleaves within) accessible sequences. The treated chromatin is optionally deproteinized and then fragmented to produce a mixture of polynucleotide fragments, wherein the mixture comprises fragments containing at least one site that has reacted with the probe (marked polynucleotide fragments) and fragments that have not reacted with the probe (unmarked polynucleotide fragments). Marked fragments are selected and correspond to accessible regions of cellular chromatin.

Fragmentation is achieved by any method of polynucleotide fragmentation known to those of skill in the art including, but not limited to, nuclease digestion (e.g., restriction enzymes, non-sequence-specific nucleases such as DNase I, micrococcal nuclease, S1 nuclease and mung bean nuclease), and physical methods such as shearing and sonication. Isolation is accomplished by any technique that allows for the selective purification of marked fragments from unmarked fragments (e.g., size or affinity separation techniques and/or purification on the basis of a physical property).

2. Methods with Enzymatic Probes

A variety of enzymatic probes can be used to identify accessible regions of chromatin. Suitable enzymatic probes in general include any enzyme that can react with one or more sites in an accessible region to, for example, modify a nucleotide within the region, thereby generating a modified product. The modification provides the basis for selection of marked polynucleotides and their separation from unmarked polynucleotides.

DNA methyltransferase enzymes (or simply methylases) are examples of one group of suitable enzymes. Of the naturally occurring nucleosides only thymidine contains a methyl group (at the 5-position of the pyrimidine ring). Bacterial and eukaryotic methylases generally add methyl groups to nucleosides other than thymidine, to form, for example, N⁶-methyladenosine and 5-methylcytidine.

Methods employing methylases generally involve contacting cellular chromatin with a DNA methylase such that accessible DNA sequences are methylated. The chromatin is optionally deproteinized and, in one embodiment, the resulting methylated DNA is subsequently treated with a methylation-sensitive nuclease to generate large fragments corresponding to accessible regions. Alternatively, or in addition, methylated chromatin or DNA is treated with a methylation-dependent nuclease (e.g., a restriction enzyme that does not cleave at its recognition sequence unless the recognition sequence is methylated) to generate small fragments comprising accessible regions and larger fragments whose boundaries comprise accessible regions. In yet another alternative, cellular chromatin is contacted with a methylase, optionally deproteinized, fragmented, and methylated DNA fragments selected using antibodies to methylated nucleotides or methylated DNA.

For example, in certain methods, the danz methylase (E. coli DNA adenine methylase), which methylates the N⁶ position of adenine residues in the sequence 5′-GATC-3′, is used. This enzyme is useful in the analysis of regulatory regions in eulcaryotic cells because adenine methylation does not normally occur in eukaryotic cells. Other exemplary methylases include, but are not limited to, AluI methylase, BamHI methylase, ClaI methylase, EcoRI methylase, FnuDII methylase, Haef methylase, HhaI methylase, HpaII methylase, Msp I metlhylase, PstI methylase, SssI methylase, TaqI methylase, dcm (Mec) methylase, EcoK methylase and Dnmtl methylase. These and related enzymes are commercially available, for example, from New England BioLabs, Inc. Beverly, Mass.

Following methylase treatment, accessible regions are identified by distinguishing methylated from non-methylated DNA. Some methods involve generating fragments of DNA and then separating those fragments that include methylated nucleotides (ie., marked fragments) from those fragments that are unmethylated (i.e., unmarked fragments). For example, in embodiments in which cellular chromatin is treated with danii methylase, methylated fragments can be isolated by affinity purification using antibodies to N⁶-methyl adenine. Bringmann et al. (1987) FEBS Lett. 213:309-315. Any affinity purification technique known in the art such as, for example, affinity chromatography using immobilized antibody, can be used.

Methylated accessible regions can also be selected and isolated based on their possession of methylated restriction sites that are resistant to cleavage by methylation-sensitive restriction enzymes. For example, subsequent to its methylation, cellular chromatin is deproteinized and subjected to the activity of a methylafion-sensitive restriction enzyme. A methylation-seisitive enzyme refers to a restriction enzymes that does not cleave DNA (or cleaves DNA poorly) if one or more nucleotides in its recognition site are methylated. Exemplary enzymes of this type include MboI and DpnII, both of which digest DNA at the sequence 5′-GATC-3′ only if the A residue is umnethylated. (Note that this is the same sequence that is methylated by dam methylase.) Since both of these enzymes have four-nucleotide recognition sequences, they generate, on average, small fragments of non-methylated DNA. Methylated regions, corresponding to areas of chromatin originally accessible to the methylase, are resistant to digestion and can be isolated, for example, based on their larger size, or through affinity methods that recognize methylated DNA (e.g., antibodies to N⁶-methyl adenine, supra). Other methylation sensitive enzymes include, but are not limited to, HpaII, and ClaI. See, in addition, the New England BioLabs 2000-01 Catalogue & Technical Reference, esp. pages 220-221 and references cited therein.

In other embodiments, preferential cleavage of methylated DNA (obtained from cellular cliomatin that has been methylated as described supra) by certain enzymes such as, for example, methylation-dependent restriction enzymes, generates small fragments, which can be separated from larger, unmethylated DNA fragments. For example, treatment of cellular chromatin with daniz methylase, followed by deproteinization and digestion of methylated DNA with DpnI (which cleaves at the 4-nucleotide recognition sequence 5′-GATC-3′ only if the A residue is methylated) will generate relatively small fragments from methylated accessible regions. These can be isolated based on size or affmity procedures, as disclosed above. In addition, the larger fragments generated by this procedure comprise the distal portions and boundaries of accessible regions at their termini and can be isolated based on size. Another methylation-dependent enzyme, which cleaves at sequence different from that recognized by Dpn I, is Mcr BC. This enzyme, as well as additional methylation-dependent restriction enzymes, are disclosed in the New England BioLabs 2000-01 Catalog and Technical Reference.

Additional enzymatic probes of chromatin structure, which can be used to idenfij accessible regions, include micrococcal nuclease, S1 nuclease, mung bean nuclease, and restriction endonucleases. In addition, the method described by van Steensel et al. (2000) Nature Biotechnol. 18:424428 can be used to identify accessible regions.

3. Methods with Chemical Probes

Another option for marking accessible regions in chromatin is to use various chemical probes. In general, these chemical probes react with a functional group of one or more nucleotides within an accessible region to generate a modified or derivatized nucleotide. Following cleavage of chromatin according to the established methods described supra, fragments including one or more derivatized nucleotides can be separated from those fragments that do not include modified nucleotides.

A variety of different chemical probes can be utilized to modify DNA in accessible regions. In general, the size and reactivity of such probes should enable the probes to react with nucleotides located within accessible regions. Chemical modification of cellular chromatin in accessible regions can be accomplished by treatment of cellular chromatin with reagents such as dimethyl sulfate, hydrazine, potassium permanganate, and osmium tetroxide. Maxam et al. (1980) Meth. Enzymology, Vol. 65, (L. Grossman & K. Moldave, eds.) Academic Press, New York, pp. 499-560. Additional exemplary chemical modification reagents are the psoralens, which are capable of intercalation and crosslink formation in double-stranded DNA.

As noted supra, once cellular chromatin has been contacted with a chemical probe and the reactants allowed a sufficient period in which to react, the resulting modified chromatin is fragmented using various cleavage methods. Exemplary techniques include reaction with restriction enzymes, sonication and shearing methods. Following fragmentation, marked polynucleotides corresponding to accessible regions can be purified from unmarked polynucleotides. Purification can be based on affinity methods such as, for example, binding to antibodies specific for the product of modification.

In certain embodiments, chemical and enzymatic probes can be combined to generate marked fragments that can be purified from unmarked fragments.

4. Methods with Binding Molecules

In certain embodiments, a molecule which is capable of binding to an accessible region, but does not necessarily cleave or covalently modify DNA in the accessible region, can be used to identify and isolate accessible regions. Suitable molecules include, for example, minor groove binders (e.g., U.S. Pat. Nos. 5,998,140 and 6,090,947), and triplex-forming oligonucleotides (TFOs, U.S. Pat. Nos. 5,176,996 and 5,422,251). The molecule is contacted with cellular chromatin, the chromatin is optionally deproteinized, then fragmented, and fragments comprising the bound molecule are isolated, for example, by affinity techniques. Use of a TFO comprising poly-inosine (poly-I) will lead to minimal sequence specificity of triplex formation, thereby maximizing the probability of interaction with the greatest possible number of accessible sequences.

In a variation of one of the aforementioned methods, TFOs with covalently attached modifying groups are used. See, for example, U.S. Pat. No. 5,935,830. In this case, covalent modification of DNA occurs in the vicinity of the triplex-forming sequence. After optional deproteinization and fragmentation of treated chromatin, marked fragments are purified by, for example, affinity selection.

In another embodiment, cellular chromatin is contacted with a non-sequence-specific DNA-binding protein. The protein is optionally crosslinked to, the chromatin. The chromatin is then fragmented, and the mixture of fragments is subjected to immunoprecipitation using an antibody directed against the non-sequence-specific DNA-binding protein. Fragments in the irnmrunoprecipitate are enriched for accessible regions of cellular chromatin. Suitable non-sequence-specific DNA-binding proteins for use in this method include, but are not limited to, prokaryotic histone-like proteins such as the bacteriophage SP01 protein TF1 and procaryotic HU/DBPII proteins. Greene et al. (1984) Proc. Natl. Acad. Sci. USA 81:7031-7035; Rouviere-Yaniv et al. (1977) Cold Spriig Harbor Symp. Quant. Biol. 42:439-447; Kimura et al (1983) J. Biol. Chem. 258:4007-4011; Tanaka et al (1984) Nature 310:376-381. Additional non-sequence-specific DNA-binding proteins include, but are not limited to, proteins containing poly-arginine motifs and sequence-specific DNA-binding proteins that have been mutated so as to retain DNA-binding ability but lose their sequence specificity. An example of such a protein (in this case, a mutated restriction enzyme) is provided by Rice et al. (2000) Nucleic Acids Res. 28:3143-3150.

In yet another embodiment, a plurality of sequence-specific DNA binding proteins is used to identify accessible regions of cellular chromatin. For example, a mixture of sequence-specific DNA binding proteins of differing binding specificities is contacted with cellular chromatin, chromatin is fragmented and the mixture of fragments is immunoprecipitated using an antibody that recognizes a common epitope on the DNA binding proteins. The resulting immunoprecipitate is enriched in accessible sites corresponding to the collection of DNA binding sites recognized by the mixture of proteins. Depending on the completeness of sequences recognized by the mixture of proteins, the accessible immunoprecipitated sequences will be a subset or a complete representation of accessible sites.

In addition, synthetic DNA-binding proteins can be designed in which non-sequence-specific DNA-binding interactions (such as, for example, phosphate contacts) are maximized, while sequence-specific interactions (such as, for example, base contacts) are minimized. Certain zinc finger DNA-binding domains obtained by bacterial two-hybrid selection have a low degree of sequence specificity and can be useful in the aforementioned methods. Joung et al. (2000) Proc. Natl. Acad. Sci. USA 97:7382-7387; see esp. the “Group III” fingers described therein.

C. Selective/Limited Digestion Methods

1. Limited Nuclease Digestion

This approach generally involves treating nuclei or chromatin under controlled reaction conditions with a chemical and/or enzymatic probe such that small fragments of DNA are generated from accessible regions. The selective and limited digestion required can be achieved by controlling certain digestion parameters. Specifically, one typically limits the concentration of the probe to very low levels. The duration of the reaction and/or the temperature at which the reaction is conducted can also be regulated to control the extent of digestion to desired levels. More specifically, relatively short reaction times, low temperatures and low concentrations of probe can be utilized.

Any of a variety of nucleases can be used to conduct the limited digestion. Both non-sequence-specific endonucleases such as, for example, DNase I, S1 nuclease, and mung bean nuclease, and sequence-specific nucleases such as, for example, restriction enzymes, can be used.

A variety of different chemical probes can be utilized to cleave DNA in accessible regions. Specific examples of suitable chemical probes include, but are not limited to, hydroxyl radicals and methidiumpropyl-EDTA.Fe(II) (MPE). Chemical cleavage in accessible regions can also be accomplished by treatment of cellular chromatin with reagents such as dimethyl sulfate, hydrazine, potassium permanganate, and osmium tetroxide, followed by exposure to alkaline conditions (e.g., 1 M piperidine). See, for example, Tullius et al. (1987) Meth. Enzymology, Vol. 155, (J. Ableson & M. Simon, eds.) Academic Press, San Diego, pp. 537-558; Cartwright et al. (1983) Proc. Natl. Acad. Sci. USA 80:3213-3217; Hertzberg et al. (1984) Biochemistry 23:3934-3945; Wellinger et al. in Methods in Molecular Biology, Vol. 119 (P. Becker, ed.) Humana Press, Totowa, N.J., pp. 161-173; and Maxam et al. (1980) Meth. Enzymology, Vol. 65, (L. Grossman & K. Moldave, eds.) Academic Press, New York, pp. 499-560.

When using chemical probes, reaction conditions are adjusted so as to favor the generation of, on average, two sites of reaction per accessible region, thereby releasing relatively short DNA fragments from the accessible regions.

As with the previously-described methods, the resulting small fragments generated by the digestion process can be purified by size (e.g., gel electrophoresis, sedimentation, gel filtration), preferential solubility, or by procedures which result in the separation of naked nucleic acid (i.e., nucleic acids lacking histones) from bulk chromatin, thereby allowing the small fragments to be isolated and/or cloned, and/or subsequently analyzed by, for example, nucleotide sequencing.

In one embodiment of this method, nuclei are treated with low concentrations of DNase; DNA is then purified from the nuclei and subjected to gel electrophoresis. The gel is blotted and the blot is probed with a short, labeled fragment corresponding to a known mapped DNase hypersensitive site located, for example, in the promoter of a housekeeping gene. Examples of such genes (and associated hypersensitive sites) include, but are not limited to, those in the genes encoding rDNA, glyceraldehyde-3-phosphate dehydrogenase (GAPDH) and core histones (e.g., H2A, H2B, H3, H4). Alternatively, a DNA fragment size fraction is isolated from the gel, slot-blotted and probed with a hypersensitive site probe and a probe located several kilobases (kb) away from the hypersensitive site. Preferential hybridization of the hypersensitive site probe to the size fraction is indicative that the fraction is enriched in accessible region sequences. A size fraction enriched in accessible region sequences can be cloned, using standard procedures, to generate a library of accessible region sequences.

In certain embodiments, regulatory regions are obtained essentially as follows:

(i) isolate intact nuclei from any cell type;

(ii) digest genomic DNA within nuclei using selected restriction enzymes and/or nucleases (e.g., DNAse I), under conditions optimized to allow, on average, a single cleavage per accessible region;

(iii) deproteinize the DNA, preferably under conditions that avoid shearing (e.g. embedding nuclei in agarose);

(iv) shear deproteinized DNA to an average size of 500 bp, e.g., by digestion with a restriction enzyme that yields DNA fragments with defined cohesive ends under controlled conditions; and

(v) clone fragments with one end cleaved by the nuclease (from step ii) and the other end cleaved during shearing (step iv) from the resulting genomic DNA pool. Clones in the resulting library comprise regulatory DNA sequences active in the cell type used.

In certain embodiments, the regulatory DNA is prepared, in part, by exposing cell nuclei to DNAseI. Preferably, the exposure to DNAseI is conducted under conditions such that the DNAseI does not substantially cleave in non-accessible regions and under conditions such that the chromatin does not shear. See, also, Examples.

Micrococcal nuclease (MNase) is used as a probe of chromatin structure in other methods to identify accessible regions. MNase preferentially digests the linker DNA present between nucleosomes, compared to bulk chromatin. Regulatory sequences are often located in linker DNA, to facilitate their ability to be bound by transcriptional regulatory molecules. Consequently, digestion of chromatin with MNase preferentially digests regions of chromatin that often include regulatory sites. Because MNase digests DNA between nucleosomes, differences in nucleosome positioning on specific sequences, between different cells, can be revealed by analysis of MNase digests of cellular chromatin using techniques such as, for example, indirect end-labeling. Since alterations in nucleosome positioning are often associated with changes in gene regulation, sequences associated with changes in nucleosome positioning are likely to be regulatory sequences.

The borders of accessible regions can be localized, if necessary, utilizing the technique of indirect end-labeling. In this method, a collection of DNA fragments obtained as described above (i.e., reaction of nuclei or cellular chromatin with a probe or cleavage agent followed by deproteinization) is digested with a restriction enzyme to generate restriction fragments that include the regions of interest. Such fragments are then separated by gel electrophoresis and blotted onto a membrane. The membrane is then hybridized with a labeled hybridization probe complementary to a short region at one end of the restriction fragment containing the region of interest. In the absence of an accessible region, the hybridization probe identifies the full-length restriction fragment. However, if an accessible region is present within the sequences defined by the restriction fragment, the hybridization probe identifies one or more DNA species that are shorter than the restriction fragment. The size of each additional DNA species corresponds to the distance between an accessible region and the end of the restriction fragment to which the hybridization probe is complementary.

2. Release of Sequences Enriched in CpG Islands

The dinucleotide CpG is severely underrepresented in mammalian genomes relative to its expected statistical occurrence frequency of 6.25%. In addition, the bulk of CpG residues in the genome are methylated (with the modification occurring at the 5-position of the cytosine base). As a consequence of these two phenomena, total human genomic DNA is remarkably resistant to, for example, the restriction endonuclease Hpa II, whose recognition sequence is CCGG, and whose activity is blocked by methylation of the second cytosine in the target site.

An important exception to the overall paucity of demethylated Hpa II sites in the genome are exceptionally CpG-rich sequences (so-called “CpG islands”) that occur in the vicinity of transcriptional startsites, and which are demethylated in the promoters of active genes. Jones et al. (1999) Nature Genet. 21:163-167. Aberrant hypermethylation of such promoter-associated CpG islands is a well-established characteristic of the genome of malignant cells. Robertson et al (2000) Carcinogenesis 21:61-467.

Accordingly, another option for generating accessible regions relies on the observation that, whereas most CpG dinucleotides in the eukaryotic genome are methylated at the C5 position of the C residue, CpG dinucleotides within the CpG islands of active genes are unmethylated. See, for example, Bird (1992) Cell 70:5-8; and Robertson et al. (2000) Carcinogenesis 21:461-467. Indeed, methylation of CpG is one mechanism by which eukaryotic gene expression is repressed. Accordingly, digestion of cellular DNA with a methylation-sensitive restriction enzyme (i.e., one that does not cleave methylated DNA), especially one with the dinucleotide CpG in its recognition sequence, such as, for example, Hpa II, generates small fragments from unmethylated CpG island DNA. For example, upon the complete digestion of genomic DNA with Hpa II, the overwhelming majority of DNA will remain >3 kb in size, whereas the only DNA fragments of approximately 100-200 bp will be derived from demethylated, CpG-rich sequences, i.e., the CpG islands of active genes. Such small fragments are enriched in regulatory regions that are active in the cell from which the DNA was derived. They can be purified by differential solubility or size selection, for example, cloned to generate a library, and their nucleotide sequences determined and placed in one or more databases. Arrays comprising such sequences can be constructed.

Digestion with methylation-sensitive enzymes, optionally in the presence of one or more additional nucleases, can be conducted in whole cells, in isolated nuclei, with bulk chromatin or with naked DNA obtained after stripping proteins from chromatin. In all instances, relatively small fragments are excised and these can be separated from the bulk chromatin or the longer DNA fragments corresponding to regions containing methylated CpG dinucleotides. The small fragments including unmnethylated CpG islands can be isolated from the larger fragments using various size-based purification techniques (e.g., gel electrophoresis, sedimentation and size-exclusion columns) or differential solubility (e.g., polyethyleneimine, spermine, spermidine), for example.

As indicated above, a variety of methylation-sensitive restriction enzymes are commercially available, including, but not limited to, DpnII, MboI, HpaII and ClaI. Each of the foregoing is available from commercial suppliers such as, for example, New England BioLabs, Inc., Beverly, Mass.

In another embodiment, enrichment of regulatory sequences is accomplished by digestion of deproteinized genomic DNA with agents that selectively cleave AT-rich DNA. Examples of such agents include, but are not limited to, restriction enzymes having recognition sequences consisting solely of A and T residues, and single strand-specific nucleases, such as S1 and mung bean nuclease, used at elevated temperatures. Examples of suitable restriction enzymes include, but are not limited to, Mse I, Tsp509 I, Ase I, Dra I, Pac I, Psi I, Ssp I and Swa I. Such enzymes are available commercially, for example, from New England Biolabs, Beverly, Mass. Because of the concentration of GC-rich sequences within CpG islands (see, above), large fragments resulting from such digestion generally comprise CpG island regulatory sequences, especially when a restriction enzyme with a four-nucleotide recognition sequence consisting entirely of A and T residues (e.g., Mse I, Tsp509 I), is used as a digestion agent. Such large fragments can be separated, based on their size, from the smaller fragments generated from cleavage at regions rich in AT sequences. In certain cases, digestion with multiple enzymes recognizing AT-rich sequences provides greater enrichment for regulatory sequences.

Alternatively, or in addition to a size selection, large, CpG island-containing fragments generated by these methods can be subjected to an affinity selection to separate methylated from unmethylated large fragments. Separation can be achieved, for example, by selective binding to a protein containing a metliylated DNA binding domain (Hendrich et al. (1998) Mol. Cell. Biol. 18:6538-6547; Bird et al. (1999) Cell 99:451-454) and/or to antibodies to methylated cytosine. Unmethylated large fragments are likely to comprise regulatory sequences involved in gene activation in the cell from which the DNA was derived. As with other embodiments, polynucleotides obtained by the aforementioned methods can be cloned to generate a library of regulatory sequences and/or the regulatory sequences can be immobilized on an array.

Regardless of the particular strategy employed to purify the unmethylated CpG islands from other fragments, the isolated fragments can be cloned to generate a library of regulatory sequences. The nucleotide sequences of the members of the library can be determined, optionally placed in one or more databases, and compared to a genome database to map these regulatory regions on the genome.

D. Immunonrecipitation

In other methods for identification and isolation of regulatory regions, enrichment of regulatory DNA sequences takes advantage of the fact that the chromatin of actively transcribed genes generally comprises acetylated histones. See, for example, Wolffe et al. (1996) Cell 84:817-819. In particular, acetylated H3 and H4 are enriched in the chromatin of transcribed genes, and chromatin comprising regulatory sequences is selectively enriched in acetylated H3. Accordingly, chromatin immunoprecipitation using antibodies to acetylated histones, particularly acetylated H3, can be used to obtain collections of sequences enriched in regulatory DNA.

Such methods generally involve fragmenting chromatin and then contacting the fragments with an antibody that specifically recognizes and binds to acetylated histones, particularly H3. The polynucleotides from the immunoprecipitate can subsequently be collected from the immunoprecipitate. Prior to fragmenting the chromatin, one can optionally crosslink the acetylated histones to adjacent DNA. Crosslinking of bistones to the DNA within the chromatin can be accomplished according to various methods. One approach is to expose the chromatin to ultraviolet irradiation. Gilmour et al. (1984) Proc. Natl. Acad. Sci. USA 81:4275-4279. Other approaches utilize chemical crosslinking agents. Suitable chemical crosslinking agents include, but are not limited to, formaldehyde and psoralen. Solomon et al. (1985) Proc. Natl. Acad. Sci. USA 82:6470-6474; Solomon et al. (1988) Cell 53:937-947.

Fragmentation can be accomplished using established methods for fragmenting chromatin, including, for example, sonication, shearing and/or the use of restriction enzymes. The resulting fragments can vary in size, but using certain sonification techniques, fragments of approximately 200-400 nucleotide pairs are obtained.

Antibodies that can be used in the methods are commercially available from various sources. Examples of such antibodies include, but are not limited to, Anti Acetylated Histone H3, available from Upstate Biotechnology, Lake Placid, N.Y.

Additional chromatin modifications of a regulatory nature, that can be identified with antibodies include, but are not limited to: global acetylation, lysine 5 acetylation, lysine 7 acetylation and lysine 9 acetylation of histone H2A; global acetylation, lysine 5 acetylation, lysine 12 acetylation, lysine 15 acetylation, lysine 16 acetylation, lysine 20 acetylation and serine 14 phosphorylation of histone H2B; global acetylation, lysine 4 methylation, lysine 9 methylation, lysine 9 trimethylation, lysine 9 acetylation, serine 10 phosphorylation, lysine 14 acetylation, arginine 26 methylation and lysine 28 methylation of histone H3; and global acetylation, lysine 8 acetylation, lysine 12 acetylation, lysine 16 acetylation and lysine 20 methylation of histone H4. Antibodies can be obtained, for example, from Abcam or Upstate Biotechnology and can comprise panels of distinct sera that distinguish among monomethylated, dimethylated and trimethylated lysine.

Identification of a binding site for a particular defined transcription factor in cellular chromatin is indicative of the presence of regulatory sequences. This can be accomplished, for example, using the technique of clromatin imniunoprecipitation. Briefly, this technique involves the use of a specific antibody to immunoprecipitate chromatin complexes comprising the corresponding antigen (in this case, the transcription factor of interest), and examination of nucleotide sequences, present in the immunoprecipitate, that are crosslinked to the antigen. Immunoprecipitation of a particular sequence by the antibody is indicative of interaction of the antigen with that sequence. See, for example, O'Neill et al. in Methods in Enzymology, Vol. 274, Academic Press, San Diego, 1999, pp. 189-197; Kuo et al. (1999) Method 19:425-433; and Current Protocols in Molecular Biology, F. M. Ausubel et al., eds., Current Protocols, Chapter 21, a joint venture between Greene Publishing Associates, Inc. and John Wiley & Sons, Inc., (1998 Supplement). After reversal of crosslinks, the released sequences can be cloned, sequenced and/or placed on an array.

As with the other methods, polynucleotides isolated from an inimunoprecipitate, as described herein, can be cloned to generate a library and/or sequenced, and/or the sequences can be placed on a nucleic acid array as described in greater detail below. Sequences adjacent to those detected by this method are also likely to be regulatory sequences. These can be identified by mapping the isolated sequences on the genome sequence for the organism from which the chromatin sample was obtained, and optionally entered into one or more databases.

E. Mapping DNase Hypersensitive Sites Relative to a Gene of Interest

A rapid method for mapping DNase hypersensitive sites (which can correspond to boundaries of accessible regions) with respect to a particular gene involves ligation of an adapter oligonucleotide to the DNA ends generated by DNase action, followed by amplification using an adapter-specific primer and a gene-specific primer. For this procedure, nuclei or isolated cellular chromatin are treated with a nuclease such as, for example, DNase I or micrococcal nuclease, and the chromatin-associated DNA is then purified. Purified, nuclease-treated DNA is optionally treated so as to generate blunt ends at the sites of nuclease action by, for example, incubation with T4 DNA Polyrnerase and the four deoxyribonucleoside triphosphates. After this treatment, a partially double-stranded adapter oligonucleotide is ligated to the DNA ends. The adapter contains a 5′-hydroxyl group at its blunt end and a 5′-extension, terminated with a 5′-phosphate, at the other end. The 5′-extension is an integral number of nucleotides greater that one nucleotide, preferably greater than 5 nucleotides, preferably greater than 10 nucleotides, more preferably 14 nucleotides or greater. Alternatively, a 5′-extension need not be present, as long as one of the 5′ ends of the adapter is unphosphorylated. This procedure generates a population of DNA molecules whose termini are defined by sites of nuclease action, with the aforementioned adapter ligated to those termini.

The DNA is then purified and subjected to amplification (e.g., PCR). One of the primers corresponds to the longer, 5′-phosphorylated strand of the adapter, and the other is complementary to a known site in the gene of interest or its vicinity. Amplification products are analyzed by, for example, gel electrophoresis. The size of the amplification product(s) indicates the distance between the site that is complementary to the gene-specific primer and the proximal border of an accessible region (in this case, a nuclease hypersensitive site). In additional embodiments, a plurality of second primers, each complementary to a segment of a different gene of interest, is used, to generate a plurality of amplification products.

In additional embodiments, nucleotide sequence determination can be conducted during the amplification. Such sequence analyses can be conducted individually or in multiplex fashion.

While the foregoing discussion on mapping has referred primarily to certain nucleases, it will be clear to those skilled in the art that any enzymatic or chemical agent, or combination thereof, capable of cleavage in an accessible region, can be used in the mapping methods just described.

F. Footprinting

Yet another method for identifing regulatory regions in cellular chromatin is by in vivo footprinting, a technique in which the accessibility of particular nucleotides (in a region of interest) to enzymatic or chemical probes is determined. Differences in accessibility of particular nucleotides to a probe, in different cell types, can indicate binding of a transcription factor to a site encompassing those nucleotides in one of the cell types being compared. The site can be isolated, if desired, by standard recombinant methods. See Wassarman and Wolffe (eds.) Methods in Enzymology, Volume 304, Academic Press, San Diego, 1999.

G. In Vitro v. In Vivo Methods

Certain methods can optionally be performed in vitro or in vivo. For instance, treatment of cellular chromatin with chemical or enzymatic probes can be accomplished using isolated chromatin derived from a cell, and contacting the isolated chromatin with the probe ill vitro. Methods that depend on methylation status can, if desired, be performed in vitro using naked genomic DNA. Alternatively, isolated nuclei can be contacted with a probe iil vivo. In certain other in vivo methods, a probe can be introduced into living cells. Cells are permeable to some probes. For other probes, such as proteins, various methods, known to those of skill in the art, exist for introduction of macromolecules into cells. Alternatively, a nucleic acid encoding an enzymatic probe, optionally in a vector, can be introduced into cells by established methods, such that the nucleic acid encodes an enzymatic probe that is active in the cell in vivo. Methods for the introduction of proteins and nucleic acids into cells are known to those of skill in the art and are disclosed, for example, in co-owned PCT publication WO 00/41566. Methods for methylating chromatin in vivo using recombinant constructs are described, for example, by Wines, et al. (1996) Chromasoma 104:332-340; Kladde, et al. (1996) EMBO J. 15: 6290-6300, and van Steensel, B. and Henikoff, S. (2000) Nature Biotechnology 18:424-428, each of which is incorporated by reference in its entirety. It is also possible to introduce constructs into a cell to express a protein that cleaves the DNA such as, for example, a nuclease or a restriction enzyme. See, for example, U.S. Pat. No. 5,792,640.

H. Deproteinization

As described above in the various isolation schemes, with certain methods it is desirable or necessary to deproteinize the chromatin or chromatin fragments. This can be accomplished utilizing established methods that are known to those of skill in the art such as, for example, phenol extraction. Various kits and reagents for isolation of genomic DNA can also be used and are available commercially, for example, those provided by Qiagen (Valencia, Calif.).

I. Hypersensitive Site Mapping to Confirm Identification of Accessible Regions

As disclosed herein, accessible regions can be identified by any number of methods. Collections of accessible region sequences from a particular cell can be cloned to generate a library, polynucleotides from the library, or portions or complements thereof, can be placed on an array, and the nucleotide sequences of the members of the library can be determined to generate a database specific to the cell from which the accessible regions were obtained. Confimmation of the identification of a cloned insert in a library as comprising an accessible region is accomplished, if desired, by mapping the cloned sequence on the genome and conducting DNase hypersensitive site mapping on cellular chromatin in the vicinity of the mapped cloned sequence. Co-localization of a particular cloned sequence with a DNase hypersensitive site validates the identity of the insert as an accessible regulatory region. Once a suitable number of distinct inserts are confimmed to reside within DNase hypersensitive sites iil vivo, larger-scale sequencing and annotation projects can be initiated. For example, a large number of library inserts can be sequenced and their map locations determined by comparison with genome sequence databases. For a given accessible region sequence, the closest ORF (open reading frame) in the genome is provisionally assigned as the target locus regulated by sequences within the accessible region. In this way, a large number of ORFs in the genome acquire one or more potential regulatory domains, the function of which can be confimmed by standard procedures.

It will be apparent that certain of the methods described herein can be used in combination to provide confimmation and additional information. For example, treatment of nuclei or cellular chromatin with a probe can be followed by any or all of: isolation of libraries of accessible DNA sequences, mapping the sites of probe reactivity and attaching one or more accessible sequences from the library to an array. Arrays of regulatory sequences are useful in a number of methods, as described below.

IV. Libraries of Accessible Polynucleotides and Sequence Determination

A. Library Formation

The isolated accessible regions can be used to form libraries of accessible regions; generally the libraries correspond to regions that are accessible for a particular cell. As used herein, the term “library” refers to a pool of DNA fragments that have been propagated in some type of a cloning vector. The libraries of regulatory domains will typically contain a single accessible DNA fragment per clone.

Accessible regions isolated by methods disclosed herein can be cloned into any known vector according to established methods. In general, isolated DNA fragments are optionally cleaved, tailored (e.g., made blunt-ended or subjected to addition of oligonucleotide adapters) and then inserted into a desired vector by, for example, ligase- or topoisomerase-mediated enzymatic ligation or by chemical ligation. To confimm that the correct sequence has been inserted, the vectors can be analyzed by standard techniques such as restriction endonuclease digestion and nucleotide sequence determination.

Additional cloning and ili vitiro amplification methods suitable for the construction of recombinant nucleic acids are well known to persons of skill in the art. Examples of these techniques and instructions sufficient to direct persons of skill through many cloning techniques are found in Berger and Kimmel, Guide to Molecular Cloning Techniques, Methods in Enzymology, Volume 152, Academic Press, Inc., San Diego, Calif. (Berger); Current Protocols in Molecular Biology, F. M. Ausubel et al., eds., Current Protocols in Molecular Biology, a joint venture between Greene Publishing Associates, Inc. and John Wiley & Sons, Inc., (1987 and periodic updates) (Ausubel); and Sambrook, et al. (2001) Molecular Cloning: A Laboratory Manual, 3rd ed., each of which is incorporated by reference in its entirety.

A variety of common vector backbones are well known in the art. For cloning in bacteria, comnion vectors include pBR322 and vectors derived therefrom, such as pBLUESCRIP™, the pUC series of plasmids, as well as λ-phage derived vectors. In yeast, vectors that can be used include Yeast Integrating plasmids (e.g., YIp5) and Yeast Replicating plasmids (the YRp series plasmids), the pYES series and pGPD-2 for example. Expression in mammalian cells can be achieved, for example, using a variety of commonly available plasmids, including pSV2, pBC12BI, and p91023, the pCDNA series, pCMV1, pMAMneo, as well as lytic virus vectors (e.g., vaccinia virus, adenovirus), episomal virus vectors (e.g., bovine papillomavirus), and retroviral vectors (e.g., murine retroviruses). Expression in insect cells can be achieved using a variety of baculovirus vectors, including pFastBac1, pFastBacHT series, pBluesBac4.5, pBluesBacHis series, pMelBac series, and pVL1392/1393, for example. Additional vectors and host cells are well known to those of skill in the art in view of the teachings herein.

The libraries formed thus represent regulatory regions from any cell type and/or subject, for example untransformed human cells and/or one or more cancer cell lines. Non-limiting examples of suitable cells from which to prepare DNA regulatory libraries described herein include primary foreskin fibroblasts (ATCC CRL-2522); white blood cells filtered from whole blood (Memorial Blood Centers of Minnesota); pooled placental cells (CHORI); skeletal myocytes (Clonetics); and MCF-7 cells, a breast carcinoma cell line (ATCC HTB-22). Any other cell type can be used, for example any of the cell types available from the ATCC.

Furthermore, because genome activity is cell-type specific, and because regulatory DNA activity correlates with that of the genome, a panel of regulatory DNA libraries from cell types from major embryonic lineages (e.g., ectoderm, endoderm, and mesodern) can be generated. Male and/or female cells are used, depending on the application, although male cells may be preferred in certain instances to ensure inclusion of Y-chromosome specific regulatory DNA.

In addition, the regulatory sequence in each clone can be virtually any length, and is preferably between about 25 bp and about 1,000 bp in length (or any value therebetween), more preferably between about 50 and about 500 bp in length (or any value therebetween), or between about 100 and 300 bp in length (or any value therebetween). As noted above, regulatory sequences can be isolated from any cell type.

The size (number of clones) in each library may vary, for example with between several hundred to a hundred thousand or more members (clones). For example, the regulatory DNA library prepared from HEK 293 cells described in the Examples included approximately 40,000 different clones.

Alternatively or in addition, such individual libraries can be combined to form a collection of libraries. Essentially any number of libraries can be combined. Typically, a collection of libraries contains at least 2, 5 or 10 libraries, each library corresponding to a different type of cell or a different cellular state. For example, a collection of libraries can comprise a library from cells infected with one or more pathogenic agents and a library from counterpart uninfected cells. Determination of the nucleotide sequences of the members of a library can be used to generate a database of accessible sequences specific to a particular cell type.

In a separate embodiment, subtractive hybridization and/or difference analysis techniques can be used in the analysis of two or more collections of accessible sequences, obtained by any of the methods disclosed herein, to isolate sequences that are unique to one or more of the collections. For example, accessible sequences from normal cells can be subtracted from accessible sequences present in virus-infected cells to obtain a collection of accessible sequences unique to the virus-infected cells. Conversely, accessible sequences from virus-infected cells can be subtracted from accessible sequences present in uninfected cells to obtain a collection of sequences that become inaccessible in virus-infected cells. Such unique sequences obtained by subtraction can be used to generate libraries and/or databases. Methods for subtractive hybridization and difference analysis are known to those of skill in the art and are disclosed, for example, in U.S. Pat. Nos. 5,436,142; 5,501,964; 5,525,471 and 5,958,738.

Analysis (e.g., nucleotide sequence determination) of libraries of accessible region sequences can be facilitated by concatenating a series of such sequences with interposed marker sequences, using methods similar to those described in U.S. Pat. Nos. 5,695,937 and 5,866,330.

B. High-Throughput Library Construction

Rapid, high-throughput construction of libraries of accessible regions can be achieved using a combination of nuclease digestion and ligation-mediated PCR. Pfeifer et al. (1993) Meth. In Mol. Biol. 15:153-168; Mueller et al. (1994) In: Current Protocols in Molecular Biology, ed. F. M. Ausubel et al., John Wiley & Sons; Inc., vol. 2, pp. 15.5.1-15.5.26. Nuclei or isolated cellular chromatin are subjected to the action of one or more nucleases such as, for example, a restriction enzyme, DNase I and/or micrococcal nuclease, and the digested DNA is purified and end-repaired using, for example, T4 DNA polymerase and the four deoxyribonucleoside triphosphates. A ligation reaction is conducted using, as substrates, the nuclease-digested, end-repaired chromosomal DNA and a double-stranded adapter oligonucleotide. The adapter has one blunt end, containing a 5′-phosphate group, which is ligated to the ends generated by nuclease action. The other end of the adapter oligonucleotide has a 3′ extension and is not phosphorylated (and therefore is not capable of being ligated to another DNA molecule). In one embodiment, this extension is two bases long and has the sequence TT, although any size extension of any sequence can be used.

Adapter-ligated DNA is digested with a restriction enzyme that generates a blunt end. Preferably, the restriction enzyme has a four-nucleotide recognition sequence. Examples include, but are not limited to, Rsa I, Hae III, Alu I, Bst UI, and Cac81. Altematively, DNA can be digested with a restriction enzyme that does not generate blunt ends, and the digested DNA can optionally be treated so as to produce blunt ends by, for example, exposure to T4 DNA Polynierase and the four deoxynucleoside triphosphates.

Next, a primer extension reaction is conducted, using Taq DNA polymerase and a primer complementary to the adapter. The product of the extension reaction is a double-stranded DNA molecule having the following structure: adapter sequence/nuclease-generated end/internal sequence/restriction enzyme-generated end/3′terminal A extension. The 3′-terminal A extension results from the terminal transferase activity of the Taq DNA Polymerase used in the primer extension reaction.

The end containing the 3′-terminal A extension (i e., the end originally generated by restriction enzyme digestion after ligation of the adapter) is joined, by DNA topoisomerase, to a second double-stranded adapter oligonucleotide containing a 3′-terminal T extension. In one embodiment, prior to joining, the adapter oligonucleotide is covalently linked, through the 3′-phosphate of the overhanging T residue, to a molecule of DNA topoisomerase. See, for example, U.S. Pat. No. 5,766,891. This results in the production of a molecule containing a first adapter joined to the nuclease-generated end and a second adapter joined to the restriction enzymne-generated end. This molecule is then amplified using primers complementary to the first and second adapter sequences. Amplification products are cloned to generate a library of accessible regions and the sequences of the inserts can be determined to generate a database. The accessible regions can be placed on an array.

In the practice of the aforementioned method, it is possible to obtain DNA fragments in which both ends of the fragment have resulted from nuclease cleavage (N—N fragments). These fragments will contain both the first and second adapters on each end, with the first adapter internal to the second. Any given fragment of this type will theoretically yield four amplification products which, in sum, will be amplified twice as efficiently as a fragment having one nuclease-generated end and one restriction enzyme-generated end (N—R fragments). Thus, the final population of amplified material will comprise both N—N fragments and N—R fragments. Amplification using only one of the two primers will yield a population of amplified molecules that is enriched for N—N fragments (which will, under these conditions, be amplified exponentially, while N—R fragments will be amplified in a linear fashion). A population of amplification products enriched in N—R fragments can be obtained by subtracting the N—N population from the total population of amplification products. Methods for subtraction and subtractive hybridization are known to those of skill in the art. See, for example, U.S. Pat. No. 5,436,142; 5,501,964; 5,525,471 and 5,958,738.

In another embodiment, cellular chromatin is subjected to limited nuclease action, and fragments having one end defined by nuclease cleavage are preferentially cloned. For example, isolated chromatin or permeabilized nuclei are exposed to low concentrations of a nuclease (e.g., DNase I restriction enzyme), optionally for short periods of time (e.g., one minute) and/or at reduced temperature (e.g., lower than 37° C.). DNase-treated chromatin is then deproteinized and the resulting DNA is digested to completion with a restriction enzyme, preferably one having a four-nucleotide recognition sequence. Any or all of the steps of nuclease treatment, deproteinization and restriction enzyme digestion are optionally conducted on DNA that has been embedded in agarose, to prevent shearing which would generate artifactual ends.

Preferential cloning of nuclease-generated fragments is accomplished by a number of methods. For example, prior to restriction enzyme digestion, nuclease-generated ends can be rendered blunt-ended by appropriate nuclease and/or polymerase treatment (e.g. T4 DNA polymerase plus the 4 dNTPs). Following restriction digestion, fragments are cloned into a vector that has been cleaved to generate a blunt end and an end that is compatible with that produced by the restriction enzyme used to digest the nuclease-treated chromatin. For example, if Sau 3AI is used for digestion of nuclease-treated chromatin, the vector can be digested with Bam HI (which generates a cohesive end compatible with that generated by Sau 3AI) and Eco RV or Sma I (either of which generates a blunt end).

Ligation of adapter oligonucleotides, to nuclease-generated ends and/or restriction enzyme-generated ends, can also be used to assist in the preferential cloning of fragments containing a nuclease-generated end. For example, a library of accessible sequences is obtained by selective cloning of fragments having one blunt end (corresponding to a site of nuclease action in an accessible region) and one cohesive end, as follows. Nuclease-treated chromatin is digested with a first restriction enzyme that produces a single-stranded extension to generate a population of fragments, some of which have one nuclease-generated end and one restriction enzyme-generated end and others of which have two restriction enzyme-generated ends. If this collection of fragments is ligated to a vector that has been digested with the first restriction enzyme (or with an enzyme that generates cohesive termini that are compatible with those generated by the first restriction enzyme), fragments having two restriction enzyme-generated ends will generate circular molecules, while fragments having a restriction enzyme-generated end and a nuclease-generated end will only ligate at the restriction enzyme-generated end, to generate linear molecules slightly longer than the vector. Isolation of these linear molecules (from the circular molecules) provides a population of sequences having one end generated by nuclease action, which thereby correspond to accessible sequences. Separation of linear DNA molecules from circular DNA molecules can be achieved by methods well known in the art, including, for example, gel electrophoresis, equilibrium density gradient sedimentation, velocity sedimentation, phase partitioning and selective precipitation. The isolated linear molecules are then rendered blunt ended by, for example, treatment with a DNA polymerase (e.g., T4 DNA polymerase, E. coli DNA polymerase I Klenow fragment) optionally in the presence of nucleoside triphosphates, and recircularized by ligation to generate a library of accessible sequences.

An alternative embodiment for selective cloning of fragments having one nuclease-generated end and one restriction enzyme-generated end is as follows. After restriction enzyme digestion of nuclease-treated chromatin, protruding restriction enzyme-generated ends are “capped” by ligating, to the fragment population, an adapter oligonucleotide containing a blunt end and a cohesive end that is compatible with the end generated by the restriction enzyme, which reconstitutes the recognition sequence. The fragment population is then subjected to conditions that convert protruding ends to blunt ends such as, for example treatment with a DNA polynierase in the presence of nucleoside triphosphates. This step converts nuclease-generated ends to blunt ends. The fragments are then re-cleaved with the restriction enzyme to regenerate protruding ends on those ends that were originally generated by the restriction enzyme. This results in the production of two populations of fragments. The first (desired) population comprises fragments having one nuclease-generated blunt end and one restriction enzyme-generated protruding end; these fragments are derived from accessible regions of cellular chromatin. The second population comprises fragments having two restriction enzyme-generated protruding ends. Ligation into a vector containing one blunt end and one end compatible with the restriction enzyme-generated protruding end results in cloning of the desired fragment population to generate a library of accessible sequences.

An additional exemplary method for selecting against cloning of fragments having two restriction enzyme-generated ends involves ligation of nuclease-treated, restriction enzyme digested DNA to a linearized vector whose ends are compatible only with the ends generated by the restriction enzyme. For example, if Sau 3AI is used for restriction digestion, a Bam HI-digested vector can be used. In this case, fragments having two Sau 3AI ends will be inserted into the vector, causing recircularization of the linear vector. For fragments having a nuclease-generated end and a restriction enzyie-generated end, only the restriction enzyme-generated end will be ligated to the vector; thus the ligation product will remain a linear molecule. In certain embodiments, E. coli DNA ligase is used, since this enzyme ligates cohesive-ended molecules at a much higher efficiency than blunt-ended molecules. Separation of linear from circular molecules, and recovery of the linear molecules, generates a population of molecules enriched in the desired fragments. Such separation can be achieved, for example, by gel electrophoresis, dextran/PEG partitioning and/or spermine precipitation. Alberts (1967) Meth. Enzymology 12:566-581; Hoopes et al. (1981) Nucleic Acids Res. 9:5493-5504. End repair of the selected linear molecules, followed by recircularization, results in cloning of sequences adjacent to a site of nuclease action.

Size fractionation can also be used, separately or in connection with the other methods described above. For example, after restriction digestion, DNA is fractionated by gel electrophoresis, and small fragments (e.g., having a length between 50 and 1,000 nucleotide pairs) are selected for cloning.

In another embodiment, regulatory regions are preferentially cloned using the unique cohesive overhang characteristic of regulatory DNA that has been cleaved with a nuclease in chromatin (e.g., a CG overhang when HpaII is used for cleavage). Nuclei or cellular chromatin are exposed to brief Hpa II digestion, and the chromatin is deproteinized and digested to completion with a secondary restriction enzyme, preferably one that has a four-nucleotide recognition sequence (e.g., Sau3A). Any or all of the steps of initial cleavage (e.g., by HpaII), deproteinization and restriction enzyme digestion are optionally conducted on DNA that has been embedded in agarose, to prevent shearing that would generate artifactual ends. Fragments containing one Hpa II end and one end generated by the secondary restriction enzyme are preferentially cloned into an appropriately digested vector. For example, if the secondary restriction enzyme is Sau 3AI, the vector can be digested with Cla I (whose end is compatible with a Hpa II end) and Bam HI (whose end is compatible with that generated by Sau 3AI), thus leading to selective cloning of Hpa II/Sau 3AI regulatory DNA fragments.

In certain embodiments, fragment of accessible DNA, obtained by any of the methods disclosed herein, can be ligated into an adapter containing a promoter (e.g., a T7 promoter, a T3 promoter or a SP6 promoter). Subsequently, the cloned regulatory DNA can be directly amplified and/or labeled for screening using the arrays described herein, using standard methods. Optionally, a biotinylated oligonucleotide adapter may be ligated to one end (e.g., the end obtained by initial cleavage in an accessible region) of a regulatory DNA fragment from a library, and the regulatory DNA precipitated using avidin. The strength of the biotin-avidin interaction allows for repeated, high-stringency washes to eliminate non-regulatory DNA from the preparations. Any known binding pair may also be used for this purpose. Similarly, the second end of the regulatory fragment (generated by the second nuclease) can be ligated using a second adapter specific to the end generated by the second nuclease. Regulatory fragments can then be amplified (e.g., by PCR) using primers specific for the two adapters. Thus, ligation of adapter oligonucleotides, as described herein, to nuclease-generated ends and/or to the ends generated by the secondary restriction enzyme, can also be used to assist in the preferential cloning of fragments.

Size fractionation can also be used, separately or in connection with the other methods described above. For example, after digestion with the secondary restriction enzyme, DNA is fractionated by gel electrophoresis, and small fragments (e.g., having a length between 50 and 1,000 nucleotide pairs) are selected for cloning.

C. Sequencing

Purified and/or amplified DNA fragments comprising accessible regions can be sequenced according to known methods. In some instances, the isolated polynucleotides are cloned into a vector that is introduced into a host to amplify the sequence and the polynucleotide then purified from the cells and sequenced. Depending upon sequence length, cloned sequences can be rapidly sequenced using commercial sequencers such as the Prism 377 DNA Sequencers available from Applied Biosystems, Inc., Foster City, Calif.

D. Analysis/Selection of Libraries

As noted above, various techniques can be used to evaluate the library and determine whether it will be used for further purposes such as to make an array. Non-limiting examples of analysis techniques include sequencing, evaluating the location of cloned fragments on the genome (e.g., in relation to DNaseI hypersites and/or genes), and/or evaluation of regulatory nature of the fragments (e.g., comparison to expression profiles, transcription factor site binding density, and/or conserved sequences relative to mouse genome). These methods may be used alone or in combination.

For example, any number of clones from any given library may be randomly selected and sequenced. Clones that fall within 500 bp of transcription start sites of known genes may be referred to as “promoter” clones based on their proximity to a transcription start site. The remaining (non-promoter) clones can be evaluated to determine the percentage of clones that co-localize with DNaseI hypersensitive sites, for example by randomly selecting non-promoter clones and mapping chromatin structure at each location by conventional indirect end-labeling. Libraries in which more than 10% of the randomly selected non-promoter clones are not derived from DNaseI hypersensitive sites are typically not selected for further manipulations and one or more additional libraries are prepared from the same cell type using different experimental conditions (e.g., lower restriction enzyme concentrations).

In addition, some or all clones in a library that lie within 10 kb of the transcription start site of known genes can be compared to the expression profile of the cell type used for regulatory DNA library preparation using any suitable technique, for example using Affymetrix equipment that allows expression-profiling from the same cells from which the regulatory DNA library is prepared.

Some or all clones (e.g., non-promoter clones) of a library can also be evaluated for transcription factor binding site density. Often, an average increase of at least 2-fold or 4-fold in the number of transcription factor binding sites per fragment, relative to bulk genomic DNA of identical GC composition, is obtained. Such evaluation can be conducted using any suitable techniques, for example, using publicly available databases such as TransFac. See, for example, Wingender et al. (1997, 2001).

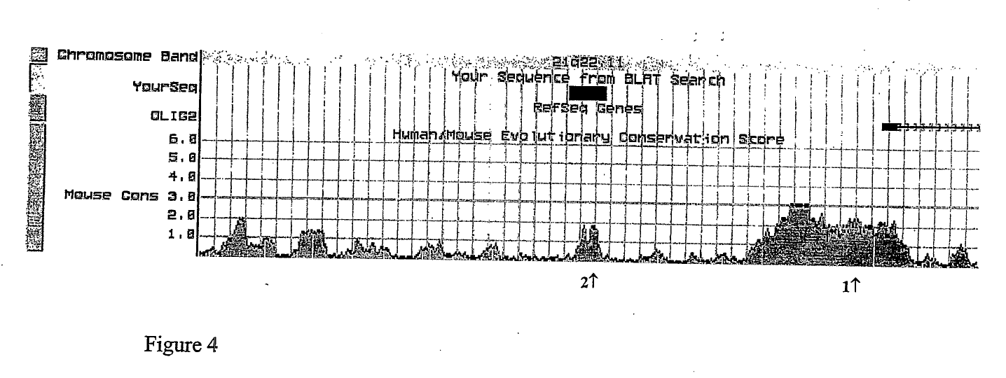

Sequence conservation, for example with other mammalian genomes such as mouse, can also be used to help evaluate the suitability of a particular library. See, also, Pennacchio et al. (2001) Nat Rev Genet 2:100-109. Sequence analysis can be readily conducted using publicly available genome analysis tools. Sequence conservation analysis is rarely used alone to identify regulatory DNA, but does provide another tool for validating the regulatory nature of the experimentally obtained DNA fragments. One, though not the only, criterion for suitability of a library is if at least about 75% of those clones that fall in mouse-human syntenic regions reside in regions of a >2.0 conservation score as defined by the UCSC Human/Mouse Evolutionary Conservation Score metric (FIG. 4).

DNA libraries that meet the test criteria may then be sequenced. Preferably, sequencing is limited to the cloned DNA fragment (e.g., about 100-500 bp). Information gathered after the initial 1,000 clones in a library have been sequenced can be further analyzed computationally to estimate library depth. Libraries predicted to contain >10,000 unique clones may then be sequenced to completion (“completion” in this case is defined as fewer than 2% new clones identified per 100 sequence reads). Sequence information can be assembled into a database with LocusID-style identifiers designating each clone by cytological location and distance from the transcription start site of the nearest gene.

Libraries generated and sequenced from different cell types (e.g., skin, blood, muscle, placenta) may also be cross-referenced to evaluate the number of shared and unique clones. For example, the total number of unique clones in the compared libraries can be assessed as well as the number of clones unique to each cell-specific library. These analyses, performed using standard techniques as described herein, can be used to assess whether a sufficiently representative number of regulatory fragments are contained in the libraries. For instance, if the total number of unique clones in the combined libraries exceeds approximately 2 per gene, further sequencing may not be necessary and the library may be deemed to be sufficiently representative of regulatory sequences of that cell type.

Libraries used to make arrays preferably include a sufficient number of clones to represent about 80% of all regulatory sequences in the genome under study. Given that a conservative estimate of the total number of regulatory DNA segments in the human genome is approximately 60,000 (ie., about 2 per gene), the libraries described herein that are used to make arrays comprising human regulatory sequences preferably represent approximately 48,000 individual regulatory DNA regions, as determined using one or more of the techniques set forth herein. In addition, libraries used in construction of regulatory arrays typically include at least 10,000 clones that are located within about 1 kb of either side of a transcription start site as measured, for example, by comparison to the human transcriptome, as defined by UniGene.

E. Library Applications

As described in detail below, the regulatory DNA libraries described herein are used to facilitate production of arrays of regulatory DNAs. In addition, the libraries themselves may be used for various applications, for example to identify unique DNA sequences for targeting of regulatory DNA binding proteins.

For example, a collection of regulatory DNA sequences is analyzed, e.g., by a computer algorithm, and stretches of DNA unique to a particular regulatory region are identified. The identified sites represent potential target sites for binding by an engineered transcription factor. Engineered transcription factors, such as zinc finger proteins (ZFPs), can be used to regulate the expression of endogenous genes in cells and animals. Furthermore, engineered ZFPs can be designed to recognize any target sequence in DNA. See, e.g., U.S. Pat. Nos. 6,511,808; 6,503,717; 6,453,242; 6,534,261; 6,599,692; and 6,607,882. Preferably, the target sequence is between about 9-18 bp.

Sequences unique to a regulatory region, as described above, are identified by any suitable method, typically involving a number of steps. For example, genomic DNA surrounding the target gene may first be identified (e.g., using BLAST searching capabilities). A selected portion of the genome surrounding the target gene (approximately 20 kilobases) can then be compared to the complete set of regDNA sequences in order to identify the subset of regDNA regions that lie within the selected region. Once identified, these regDNA regions would each be parsed back against the entire regDNA database to find stretches of approximately 9-18 bp of unique sequence. The sequences identified as unique would be the preferred target sites for binding of a regulatory DNA binding protein. It should be noted that the DNA binding protein designed to recognize the unique target site may not recognize the entire unique sequence, for example ZFPs that recognize 9 base pair sequences may be used in certain instances.

V. Arrays

Regulatory sequences present in libraries obtained as described above can be placed on an array or, alternatively, polynucleotide probes may be designed to represent the clones of the libraries and the probes then ordered into one or more arrays. Preferably, unique sequence signatures (e.g., “regDNA tags”) are used, probe sets for each regDNA tag are designed, and the probe set is synthesized on or attached to a substrate array (e.g., regDNA chip) using standard techniques.

Methods for the construction of polynucleotide arrays are known in the art. In certain methods, each polynucleotide on the array is synthesized ini situ at a predetermined location on the array. See, for example, U.S. Pat. Nos. 5,143,854; 5,489,678; 5,744,305 and 6,600,031. In other methods, different pre-synthesized polynucleotides are attached to a substrate at individual, predetermined locations to form an array. See, for example, U.S. Pat. Nos. 5,807,522 and 6,110,426. Arrays can comprise DNA, RNA or other modified or synthetic polynucleotides. In addition, the arrays can comprise single-stranded polynucleotides, double-stranded polynucleotides, or any combination. Arrays comprising single-stranded polynucleotides can be used, e.g., for hybridization to other polynucleotides. Arrays comprising double-stranded polynucleotides can be used, e.g. to assess binding of proteins to sequences on the array. Methods for production of arrays comprising double-stranded polynucleotides are disclosed, for example, in U.S. Pat. Nos. 6,326,489 and 6,548,021 and in WO 02/18648.

Members of certain of the libraries prepared as described above typically contain DNA fragments that identify, via their nuclease-generated end, the precise location of a regulatory DNA element. The other end of the DNA fragment, typically located on the order of about 500 bp away, is generated, e.g., by a restriction enzyme during controlled shearing. As a consequence, each specific fragment contains approximately 100-300 bp of a stretch of regulatory DNA, as well as 100-400 bp of immediately adjacent sequence. Thus, the arrays described herein may include the entire fragments obtained from the library, the regulatory stretch alone or the adjacent sequence alone (or probes designed to recognize, e.g., by sequence complementarity, these fragments, regulatory stretches and or polynucleotides adjacent to the regulatory sequences). For example, if a particular regulatory DNA region of a fiagment is deemed unsuitable for interrogation in the context of the entire array, the adjacent DNA of the fragment can be used as the basis for probe set design. Preferably, the tag sequence of the fragment (to which a probe may be designed) is less than about 300 bp away from the end of the regulatory DNA sequence. A probe (or probe set) that is approximately 300 bp away from a putative, site of transcription factor binding is quite acceptable for determining whether the factor is bound there, e.g., by chromatin immunoprecipitation (ChIP), because the DNA fragments obtained in a ChIP experiment are typically approximately 500 bp long.

The sequences (or probes) on each array can include regulatory sequences from any number of cell types and/or subjects (with or without various treatment protocols). For instance, an exemplary microarray, termed “the master epichip,” includes regulatory sequences that are broadly representative and inclusive of most or all of the complement of such DNA regulatory elements present in a genome, e.g., a human genome. Typically, a “master epichip” includes regulatory sequences (or probes thereto) identified as described above from a broad panel of available primary human tissues and/or cell lines including, but not limited to, whole blood nucleated cells, bone marrow, placenta, fibroblasts, stem cells (embryonic and adult), myocytes, cancer cell lines covering a wide range of tumor types (by tissue of origin, histology, propensity to metastasis, etc.), and cells challenged with a variety of environmental stimuli (heat shock, DNA damage, cell cycle arrest, growth stimulus, ECM culture substratum, etc.). Generally, a master epichip allows for the simultaneous interrogation of at least 60,000 regDNA elements. Such master epichips can be made from accessible sequences of any animal or plant (e.g., buffalo chip, potato chip). Additionally, master epichips comprising regulatory sequences of infectious agents, such as bacteria, viruses and single-celled eukaryotes, can be prepared.

Other exemplary arrays will include regulatory sequences derived primarily or totally from one or more particular tissues or cell types. This type of array, termed a “tissue epichip,” typically includes regulatory sequences (or probes thereto) identified from a particular tissue or cell type, for example, brain, liver, heart, lung, muscle, connective tissue, breast, prostate, immune tissue, etc or tumors thereof. To give but a single example, a hematological epichip would contain regDNA prepared from whole-blood sorted nucleated cells and bone marrow, and, in some embodiments, a defined panel of cells derived from hematological malignancies, such as leukemias. Generally, a tissue epichip allows for the interrogation of more than 20,000 regDNA elements.

Yet another exemplary array is termed “a state-specific epichip” and comprises a microarray of regDNA corresponding to the panel of regDNA elements in a given cell or tissue type that are responsive to a particular environmental or developmental stimulus. The rnicroarray is assembled by subjecting the tissue/cell type of interest to one or more stimuli, for example, administration of a hormione, environmental insult such as DNA damage or other stress, etc.; and subsequently preparing regDNA as described above from treated and untreated samples. In additional embodiments, regDNA is prepared from diseased and normal cells, infected and uninfected cells, cells from different tissues, or cells at different stages of development. Known subtractive procedures such as subtractive hybridization and representational difference analysis (RDA) may be used to identify regDNA elements that are uniquely represented in one or the other of the samples being compared. See, for example, Lisytsin et al. (1993) Scieince 259:946-951; Lisytsin et al. (1995) Methods in Enzymology 254:291-304 and U.S. Pat. Nos. 5,436,142; 5,501,964 and 5,958,738. Such unique sequences are then placed on an array.

It is evident that the arrays of various dimensions can be used. In certain embodiments, the regulatory sequences are prepared in microarrays, the term given to sets of miniaturized chemical reaction areas that may also be used to test DNA fragments, antibodies, or proteins and the like. Microarrays, and preparation of these microarrays, are described extensively in the literature, for example in U.S. Pat. No. 6,576,424 and references cited therein. See also Horak et al. (2002) Proc. Natl. Acad. Sci. USA 99:2924-2929 and McGall et al. (2002) Adv. Biochem. Eng. Biotechnol 77:2142. An array of regulatory sequences, wherein the sequences present on the array are identified by virtue of their accessibility in cellular chromatin, can comprise any number of sequences, e.g., two or more. In certain embodiments, the one or more arrays as described herein contain a total of more than 50,000 regulatory DNA sequences (or probes thereto) identified as described above, for example between about 20,000 and 100,000 sequences or any value therebetween. In certain embodiments, approximately 65,000 regulatory DNA elements, identified and isolated based on accessibility in cellular chromatin, are ordered into one or more arrays. Further, the particular sequences making up the array can be from the same cell type, including but not limited to, normal cells from the same or different organs/structures of a subject, diseased cells from the same or different organs/structures of a subject, or cells treated with one or more drugs such as small molecules (with a molecular weight less than 10 kD), antibodies, or the like from the same or different organs/structures of a subject. Alternatively, a single array may contain regulatory sequences from multiple different cell types and/or subjects.

Methods for preparation of nucleic acids and/or proteins to be contacted with an array (e.g., amplification, labeling) and methods for detection of nucleic acid or protein bound at a particular site on an array are known in the art and involve, for example, PCR, fluorescent labeling and use of conjugated binding pairs such as avidin and biotin (e.g., detection of a biotinylated polynucleotide with an avidin-conjugated antibody or flurophore. Secondary antibodies conjugated to detectable molecules or enzymes can be used for signal amplification.

VI. Applications

The regulatory DNA arrays (or “regDNA chip” or “epichip”) can be used for a variety of purposes. Non-limiting examples of such applications are set forth below.

A. Identification of Binding Sites for Human Regulatory Proteins

In yeast, chromatin immunoprecipitation-based methods have long been used to identify regulatory sequences that are bound by particular transcription factors and other DNA-binding proteins. As shown in the first four steps of the flowchart of FIG. 5, chromatin immunoprecipitation generally involves (1) subjecting living cells to conditions which result in protein-DNA crosslinking, thereby covalently linking DNA-binding proteins to the sequences to which they are bound in the cell; (2) shearing chromatin to a small size; (3) immunoprecipitating the sheared, crosslinked chromatin using an antibody against the protein of interest, under conditions such that the DNA chemically crosslinked to the protein will co-precipitate; and (4) reversing the crosslinks to obtain the bound DNA for further analysis. Typically, the DNA portions of the immunoprecipitated crosslinked cellular chromatin are then amplified, optionally labeled, and hybridized to a microarray containing the intergenic DNA from the yeast genome. This type of analysis of chromatin immunoprecipitated DNA on an array is also knowvn as “ChIP on a chip,” because it analyzes DNA output from a chromatin immunoprecipitation (CHIP) on a regulatory DNA microarray, or chip. DNA that subsequently yields a high signal on the microarray represents sequences that were bound iii vivo by the protein of interest in the native nuclear context.

As noted above, since all yeast regulatory sequences are intergenic, arrays representing yeast sequences can be readily obtained simply by constructing an array of intergenic sequences, and such arrays can be used to detect the targets of any given yeast transcription factor, for example one that has been subject to chromatin immunoprecipitation. Wyrick et al., above and FIG. 5. However, for the complex human genome, “ChIP on a chip” cannot be conducted, as in yeast, by hybridizing DNA obtained from a ChIP to an array of intergenic sequences, because the vast amount of intergenic DNA in the human genome precludes the construction of a single chip (or even a small number of chips) containing the entire complement of human intergenic DNA. Consequently, analysis of regulatory protein binding sites in the human genome is currently limited to individual small stretches of the genome (Horak et al. (2002) Proc Natl Acad Sci 99:2924-2929; Martone et al. (2003) Proc. Natl. Acad. Sci. USA 100: 12,247-12,252); small subsets of gene promoters (Ren et al. (2002) Genes Dev 16:245-256; or computationally identified CpG-rich stretches of uncertain regulatory relevance (Weinmann et al. (2002) Gene Dev 16:235-244).

Furthermore, certain experiments have revealed binding of regulatory factors to cellular chromatin that appears to be spurious and not related to any regulatory process, indicating that it is. impossible to use a whole-genome microarray to determnine whether or not iii vivo binding of a regulator to a particular stretch is relevant to some regulatory process (Urnov (2003) J. Cell. Biochem. 84: 684).

The methods described herein allow the isolation, from among the large amount of intergenic DNA in the human genome, of only those sequences which serve a regulatory function; thereby making it possible, for the first time, to prepare a microarray of human regulatory sequences. In addition to intergenic regulatory sequences, regulatory sequences located within genes are also obtained. Accordingly, the arrays produced as described herein make possible “ChIP on a chip” to identify the direct in vivo targets, in the human genome, of any regulatory factor of interest. Moreover, and in contrast to previous methods, all binding detected in a ChIP assay, and further analyzed (by ChIP on a chip) using a regDNA array, is relevant to regulation

The generation and use of regDNA chips to map human transcriptional regulatory netw“rorks provides a unique opportunity to develop effective therapeutics for virtually every gene-based disease. For instance, as detailed in Example 4 below, ChIP on a regDNA chip analysis of targets of estrogen receptor will allow for the development of more clinically effective selective estrogen receptor modulators (SERMs), for example for treating breast cancers. See, also, Ibrahim et al. (1999) Surg Oncol 8:103-123. Similarly, chronic pain, which can be caused by transcriptional upregulation of pain receptors in certain cells, affects approximately 50 million Americans. Cox et al. (2002) Expert Rev Neurotherapeutics 1:81-91. Using the methods described herein, active regulatory sequences unique to those cells can be isolated and placed on an array which can be used to identify transcriptional regulatory molecules in the cells, thereby helping to identify the currently unknown nature of the lesion in this transcriptional regulatory network.

B. Identification of Sequence Targets

The arrays and methods described herein can be used to identify the sequence targets and binding locations of natural or synthetic DNA binding proteins (e.g., transcription factors, replication factors, recombination factors, etc) and other DNA-binding molecules (e.g., oligonucleotides, minor groove binders, antibiotics, chemotherapeutics). Furthermore, proteins tested by this method and shown to bind regulatory sequences associated with genes misregulated in disease are potential targets for therapeutic intervention. By using proteins derived from normal and/or diseased tissues, one can derive a functional link between a particular protein and its role in regulation of genes in the normal or disease state in the cell.

A protein preparation is derived from any number of potential sources. The protein preparation may be derived from normal or diseased cells or tissues. The protein preparation may be derived by expression of the gene encoding the protein in a heterologous gene expression system (E. coli, yeast, insect cells, or mammalian cell culture, for example) and optionally at least partly purified from this source. The protein may be synthesized artificially using standard protein synthesis techniques.

To identify regulatory sequences to which a protein binds, the protein preparation is put into contact with the DNA on a regDNA chip and allowed to bind. The chip can contain double-stranded or single-stranded DNA, depending on the binding properties of the protein. The protein can be labeled with any detectable label prior to, or after, contact with the array and location(s) where the protein preparation has bound can be identified. For example, the protein can be labeled with a fluorescent tag, or a fluorescently-labeled antibody to the protein can be used for detection. Alternatively, a detectable label can be attached to the DNA bound to the array; in this case, a loss of signal at one or more particular sites on the array indicates the presence of bound protein. Such DNA labels can include intercalating dyes such as ethidium bromide and SYBR Green. In additional embodiments, the nucleic acid (or polypeptide) can be labelled with a fluorescent tag, and/or a nucleic acid (or polypeptide) binding molecule can be labelled with biotin, so that an enzyme conjugate such as streptavidin-horse radish peroxidase (HRP), that catalyses an optically detectable change in a substrate (different from the fluorescent tag) can be used.

In addition, the genomic locations of the regulatory sequences bound by the protein can be readily evaluated (e.g., by identifying the regulatory sequences on the chip that are bound by the protein and searching for homology to those sequences in the human genome sequence), thereby providing an indication of which genes the protein regulates and indicating farther possible therapeutic targets. Using conventional transcriptional regulation assays, the protein can be further tested for its ability to regulate the gene(s), thereby confimming the identity of potential target genes and/or protein targets for therapeutic intervention.

C. RegDNA Profiling

An array (e.g., epichip) prepared as described above may be also used to determine the spectrum of active regDNA elements in a given cell or cell population. For example, a regulatory DNA library is obtained as described above, its sequences are amplified, amplified sequences are labeled with any suitable label, and the labeled, amplified sequences are hybridized to an array (e.g., a master epichip or a tissue epichip as descnbed above). In this way, active regDNA sequences in any selected cell or tissue type can be determined. This knowledge can then be used to determine which transcription factors may be acting in those cell types, for example, by searching the sequence of the regDNA for transcription factor binding sites and/or by mapping the active regulatory sequences onto the genome, identifying genes adjacent to the mapped regulatory sequences, and comparing those genes to the cell's transcriptome determined by genome-wide expression profiling. Transcription factors that are uniquely active in a particular cell type provide insight into pathways for potential therapeutic intervention in various disease processes.

D. Chromatin Epigenome Profiling

The arrays described herein can also be used to determine the state of histone modification (“the histone code”) at the regDNA elements in any given cell type(s). For example, chromatin inimunoprecipitation is performed (as described above) using an antibody that recognizes a particular covalent chromatin modification (e.g., histone H3 methylated on lysine 9). The immunoprecipitated DNA sequences are then hybridized to a regDNA array. Sites on the array to which immunoprecipitated DNA hybridizes represent regulatory sequences located in or adjacent to nucleosomes bearing the particular chromatin modification of interest.

In addition, data from chromatin epigenomic profiling (e.g., genes that are the direct targets of histone modifiers such as the human enhancer of zeste) can be compared between cells that overexpress the histone modifier and cells that lack it. Typically, an increased signal from modification of interest over a given DNA stretch is indicative of direct action by the modifier over this DNA stretch.

E. Chromatin-Based Toxicity Profiling

The arrays described herein also find use in evaluating the effects of a compound or treatment on a cell (e.g., toxicity, stress, etc.). For example, regDNA populations in treated cells can be isolated and characterized, and compared to those in untreated cells, if desired. Additionally, regDNAs prepared from treated cells can be hybridized to a regDNA array (epichip) as described herein to determine genes in the treated cell that are active (based on proximity to regDNA sequences isolated from the cell) in the treated cell, the histone code in the treated cell, etc. Subtractive hybridization and/or difference analysis (see above) can be used to determine regulatory sequences and genes that are preferentially activated in treated cells, compared to untreated cells.

In additional embodiments, the effect of a molecule (e.g., toxin, drug, small molecule with molecular weight less than about 10 kD) on the binding of one or more proteins to regulatory sequences can be assessed, either iii vitro or iii vivo. For example, a purified or partially purified protein can be assessed for its spectrum of binding to a double-stranded regDNA chip, in the presence and absence of a compound. For iii vivo analyses, cells can be exposed to a compound, followed by “ChIP on a chip” analysis (see above) for a DNA-binding protein of interest, to determine whether the compound alters the binding properties of the protein.

F. SNP-Epichip

Single nucleotide polyniorphisms (SNPs) are stable, bi-allelic sequence variants that are distributed. throughout the genome, which are currently assayed using a variety of high-throughput automated methods. See, e.g., Mullikin et al. (2000) Nature 407:516-520. Haplotypes are collections of linked SNPs. Using the methods and compositions described herein, SNPs and haplotypes in regulatory sequences can now be identified in any given individual. In these embodiments, regDNA is typically prepared from cells (either pooled cells or a specific cell type) or from a selected individual and hybridized to an epichip as described herein under conditions that allow SNP interrogation. Such conditions can include high stringency and/or the use of functional groups and/or nucleotide analogues that facilitate single-nucleotide mismatch discrimination. See, for example, U.S. Pat. Nos. 5,801,155; 6,127,121; 6,312,894; 6,485,906; and 6,492,346.

G. MicroRNA Validation

Short non-coding RNAs (microRNAs or miRNAs) are known to regulate cellular processes including development, heterochromatin formation, and genomic stability in eukaryotes and have been studied using available array technology. Krichevsky et al. (2003) RNA 9(10):1274-81. However, using the regDNA arrays described herein now allows the functional relevance of microRNAs to be determined, for example, by preparing a microRNA population from a cell, reverse-transcribing the RNA into cDNA, labeling the cDNA, and hybridizing the micro-cDNA to a regDNA epichip as described herein. alternatively, the microRNA can be labeled directly and used for hybridization. RegDNA elements that yield signal may correspond to microRNAs transcribed from accessible regions of chromatin.

H. Drug Discovery

Since diseased cells will typically have different genes active than a non-diseased cells, analysis of regulatory DNA is particularly applicable to drug discovery. Indeed, the arrays and methods described herein can pinpoint the differently active genes in diseased cells and this knowledge can be used to identify therapeutic targets. Non-limiting examples of diseases that can be addressed using the compositions and methods described herein include cancers of various types, chronic pain, chronic pulmonary obstruction, diabetes, ischeniic heart disease, neuropathy, coronary artery disease, peripheral arterial disease, asthma, rheumatoid arthritis, endocrine disorders, bacterial infections and viral infections.

The arrays and methods described herein greatly simplify the search and design of drugs for any disease state. For example, using the arrays and methods described above, the regulatory DNA subset active in a given cell type can be determined, for example regDNA that are aberrantly active (i.e., accessible) in individuals with at least one disorder (e.g., cancer, chronic pain, etc.). Computational analysis of these aberrantly accessible elements (e.g., regDNAs located proximally to pain receptor genes) will help identify genes whose expression is misregulated, leading to identification of the relevant regulatory proteins. Such regulatory proteins, as well as the genes they regulate, are targets for therapeutic intervention. See, e.g., Sieweke et al. (2000) Methods Mol Biol 130:59-77.

1. Identification of Genes in a “Pre-Activation” State

Expression profiling methods utilize arrays of cDNAs or cDNA-specific oligonucleotides to provide information on genes that are expressed in a cell under a particular set of conditions. See, e.g., Wyrick et at. (2002) Curr. Opin. Genet. Devel. 12: 130-136. However, transcriptional activation is a multi-step process, and includes steps that precede the production of a mRNA, which is the endpoint of an expression profiling assay. Isolation of regulatory sequences, as described herein, can identify genes that have achieved a “pre-activation” state, in which their regulatory sequences have become accessible, but transcription initiation has not yet occurred. such pre-active genes may become active subsequent to a secondary stimulus, or after passage of time. Comparison of a regulatory sequence profile with an expression profile, for a given cell or tissue, allows distinction between genes that are actively transcribed and genes that are capable of being transcribed, and distinguishes both types from inactive genes.

J. Kits

The present disclosure also includes kits for obtaining information regarding regulatory DNAs, disease, drugs, transcription pathways, etc. In certain embodiments, the kits comprise one or more of the arrays, regulatory DNAs, probes, combinations thereof, etc., described herein. For example, one exemplary kit will include at least one array that allows identification of direct genomic targets of transcription factors while another kit includes at least one array(s) for identifying the subset of regulatory DNA elements active in a given cell type. The kits described herein may also include one or more of the following: instructions, ancillary reagents or equipment, etc.

### EXAMPLES

The following examples are illustrative of but do not limit the present disclosure:

### Example 1

### Preparation of Regulatory DNA Library from HEK 293 Cells

Human embryonic kidney cells (HEK 293) were cultured in DMEM (Dulbecco's modified Eagle medium) supplemented with 10% fetal bovine serum in a 5% CO₂ incubator at 37° C. Cells were grown to 60% confluence, at which point nuclei were isolated according to the method of Archer et a. (1999) Meth. Enzymol. 304:584-599. Briefly, the plate was rinsed with PBS, cells were detached from the plate and washed with PBS, then homogenized (Dounce A) in 10 mM Tris-Cl, pH 7.4, 15 mM NaCl, 60 mM KCl, 1 mM EDTA, 0.1 mM EGTA, 0.1% NP40, 5% sucrose, 0.15 mM spermine and 0.5 mM spermidine at 4° C. Nuclei were isolated from the homogenate by centrifugation at 1,400×g for 20 min at 4° C. through a cushion of 10 mM Tris-Cl, pH 7.4, 15 mM NaCl, 60 mM KCl, 10% sucrose, 0.15 mM spermine and 0.5 mM spermidine.

Pelleted nuclei were resuspended, to a concentration of 2×10⁷ nuclei per ml, in 10 mM HEPES, pH 7.5, 25 mM KCl, 5 mM MgCl₂, 5% glycerol, 0.15 mM spermine, 0.5 mM spermidine, 1 nM dithiothreitol, 0.5 mM phenylmethylsulfonylfluoride (PMSF) and warmed to 37° C. for 30 sec. Hpa II (New England Biolabs, Beverly, Mass.) was added to a final concentration of 10,000 Units/ml and the mixture was incubated at room temperature for 5 min. The reaction was stopped by addition of EDTA to 50 mM.

An equal volume of 1% low-melting point agarose in 1×PBS warmed to 37° C. was then added, and the mixture was aspirated into the barrel of a 1 ml tuberculin syringe and incubated at 4° C. for 10 min. The agarose plugs were then extruded from the syringe and incubated for 36 hrs. with gentle shaking at 50° C. in 5 ml of 0.5 M EDTA, 1% SDS, 50 μg/ml proteinase K. The plugs were washed 3 times with 5 ml of 1×TE (pH 8.0) buffer, then incubated for 1 hr. at 37° C. in 1×TE with 1 mM PMSF, followed by two more washes with 1×TE. The plugs were placed in 2 ml of Sau3AI reaction buffer for 30 min. on ice to allow equilibration. Sau3AI was then added to 2000 units/ml and the plugs incubated with gentle shaking for 16 hrs. at 37° C. The plugs were sliced with a razor blades and slices were placed in the well of a 0.8% agarose gel in 1×TAE. The gel was run at 50V for 8 hrs., stained with SYBR-Gold, and visualized on a Dark Reader transilluminator.

Fragments having an average size of between 50 and 1000 nucleotide pairs were purified from the gel by a Qiagen gel extraction kit. The fragments purified from the gel are a mixture of San 3AI fragments (i.e., fragments having two Sau 3AI ends) and fragments having one Sau 3AI end and one Hpa II-generated end. The latter category of fragments is enriched for sequences accessible in chromatin. These fragments were preferentially cloned as follows.

The resulting population of DNA fragments was inserted into pBluescript II KS that had been digested with Bam HI and Cla I, under standard conditions. Under these conditions, Hpa II ends were inserted into the Cla I site and the Sau 3AI ends were inserted into the Bam HI site. Approximately 40,000-50,000 clones were obtained.

### Example 2

### Analysis of Selected Clones

Approximately 1% (405) of the clones of the HEK library prepared as described above were used to determine four parameters: percentage of sequences corresponding to DNaseI hypersites; genomic locations of the cloned sequences; determination of regulatory properties; and proportion of unique clones.

A. Clones Corresponding to DNaseI Hypersensitive Sites

The fraction of clones in the library that correspond to DNAse I hypersensitive sites (as opposed to, e.g., randomly sheared fragments) was tested using a pool of 10 clones randomly selected from the 405 chosen for analysis. The clones in the library were isolated based on their accessibility to nucleases within cellular chromatin. Because of the massively parallel nature of such isolation, it was important to prove by an independent method that the clones isolated truly correspond to accessible regions of cellular chromatin, e.g., DNAse I hypersensitive sites. See, for example, Gross and Garrard (1988) Annu Rev Biochem 57, 159-197. To obtain confimmation that the cloned sequences were obtained from accessible regions of cellular chromatin, the sequences of the ten clones were mapped on the genome, and the chromatin structure of the regions to which they mapped was determined (FIG. 2).

To map the cloned sequences on the genome, the human genome sequence was searched, using each of the sequences as input. For each clone, a unique location on the genome was obtained. For each of these locations, a diagnostic restriction enzyme was selected, which yielded a restriction fragment spanning the area of the genome to which the clone mapped. DNase I hypersensitive site analysis (Wu (1980) Nature 286: 854-860) was then conducted in that area of the genome. Accordingly, nuclei were isolated from HEK 293 cells, treated with DNAse I, DNA purified from DNase-I treated nuclei was subjected to digestion with the diagnostic restriction enzyme, and the locations of DNAse I hypersensitive sites were identified by indirect end-labeling (Wu, supra). For 9 out of the 10 clones, the DNA stretch in the genome identified by the clone resided in a DNAse I hypersensitive site in vivo. Four examples are provided in FIG. 2. Note that the lanes denoted “M” in FIG. 2 represent DNA digested with the diagnostic restriction enzyme and a marker restriction enizye, whose recognition sequence was within the diagnostic restriction fragment, close to the area to which the clone mapped, thereby providing a reference point on the gel. These results confimm that the methods described herein produce nuclease cleavage in non-hypersensitive areas only about 10% of the time, irrespective of the genomic location of the clone (see below)

B. Genomic Locations of Clones with Respect to Transcription Units

The genomic distribution of sequences represented in the clones was evaluated, with respect to the locations of known transcription units, to determine what fraction of the clones identified novel regulatory DNA elements and what fraction fell into already identified regions, such as core promoters.

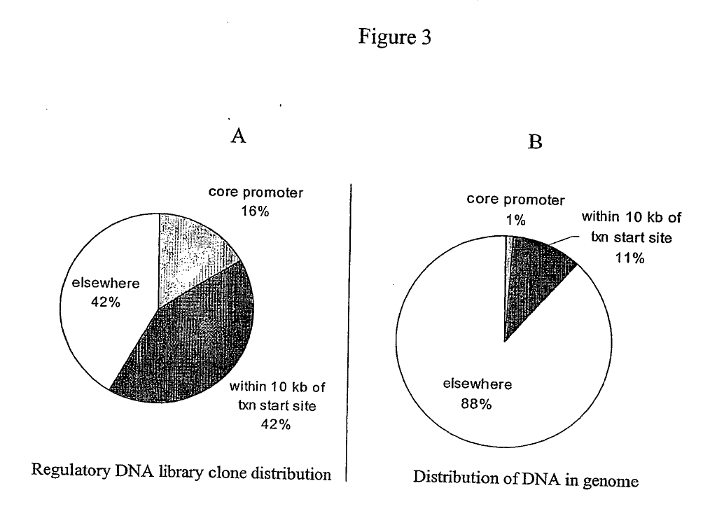

Certain of the cloned sequences were located in gene promoters (example shown in FIG. 2A). However, this analysis also revealed that clones mapped to sites well upstream of a transcription start site (e.g., FIG. 2B), 20 kb downstream of a transcription startsite (e.g., FIG. 2C) and as far as 150 kb away from the nearest known gene (e.g. FIG. 2D). Subsequently, a broader analysis of genomic location of the 405 clones randomly isolated from the regulatory DNA library was undertaken. A key prediction of a regulatory DNA isolation project is that a considerable proportion of the clones should derive from known regulatory DNA elements. A BLAST algorithm was used to evaluate the location of the 405 clones relative to the transcription start site of 35,000 annotated genes in the human genome. As shown in FIG. 3, none of the clones derived from repetitive DNA elements, which encompass about 50% of the human genome.

When the locations of the clones were compared to known transcription startsites, 58% of the clones in the library map to within 10 kb of a known transcription startsite (compared to only 12% of the human genome which lies within 10 kb of a known transcription startsite). Approximately 16% of the randomly chosen clones (66 out of 405) fell within the core promoter of known genes. The remaining 84% fell outside core promoter regions, a finding consistent with observations made on those few well-studied loci in the human genome, including the β-globin and SCL regions, in which regulatory DNA has been comprehensively experimentally mapped, and where a considerable majority of such elements was found to lie outside of the core promoter region. Bulger et al. (2002) Curr Opin Genet Dev 12:170-177; Gottgens (2000) Nat Biotechnol 18:181-186. Thus, the procedures described herein provide remarkable selectively for regulatory DNA and, in addition, identify regulatory sequences that cannot be identified computationally (e.g,. the 84% of clones that do not map to core promoter regions) but which are located in DNAseI hypersensitive sites (as shown in FIG. 2) and therefore represent boizafide regulatory DNA.

C. Repulatory Properties

The relevance, to genome regulation, of the isolated accessible sequences was evaluated to ascribe actual regulatory properties to the fragments, using criteria such as density of transcription factor binding sites, conservation in genomes of other mammals, location relative to genes known to be active in humnan kidney cells, etc. In particular, to independently confimm that the non-promoter DNA sequences were regulatory DNA, three well-established criteria for regulatory DNA were evaluated, essentially as described in Pennacchio et al. (2001) Nat Rev Genet 2:100-109, including: (1) sequence conservation between the mouse and human genomes; (2) enrichment of transcription factor binding sites; (3) location close to active genes.

As shown in FIG. 4, approximately 75% of the non-promoter, non-coding clones are located in short sequence stretches that are conserved between the mouse and human genome, representing an enormous enrichment over what would have been expected based on the overall degree of non-coding conservation of DNA sequence between the mouse and human genomes.

The isolated accessible DNA sequences are enriched relative to bulk DNA in known transcription factor binding sites. Pennacchio, above. Multiple chosen non-promoter sequences were analyzed using the publicly available TransFAC database. Wingender et al. (2001) Nucl Acid Res 29:281-283. On average, non-promoter clones had an approximately 3-fold greater number of transcription factor binding sites per 100 bp than a randomly chosen DNA sequence of identical GC-content.

Chromatin remodeling (e.g., accessibility) at regulatory DNA is known to correlate with level of gene activity. Accordingly, the 235 clones derived from within 10 kb of the start site of known genes were analyzed with respect to the activity of their gene neighbor in HEK 293 cells, using an Affymetrix GeneChip® designed for this purpose. Approximately 75% of the regulatory DNA clones were adjacent to (i.e., within 10 kb on genes that are scored as being active in HEK 293 cells by GeneChip® analysis.

D. Proportion of Unique Sequences Cloned

To determine whether the library represents a comprehensive sampling of accessible sequences in cellular chromatin, the 405 clone sequences were compared to each other in terms of their genomic location. Each clone identified a distinct location in the genome, indicating that, at least in the 405 clones chosen, there was no skew towards a particular genomic location that is preferentially accessible within cellular chromatin. Furthermore, to determine whether the library is skewed in terms of containing known regulatory DNA sequences, the genornic locations of the clones were compared to transcription start sites of known genes. According to this analysis, ˜20% of the clones identified locations within 1 kb of the transcription start sites of known genes.

These results demonstrate that at least 80% of the DNA fragments in the library correspond to genome regulatory elements that cannot be comprehensively identified using any other computational or experimental technique available. The relatively large proportion of non-promoter regulatory DNA elements active in HEK 293 cells is in accord with the literature. Pennacchio et al. (2001) Nat Rev Genet 2:100-109.

In sum, the massively parallel isolation of regulatory DNA from human cells described herein result in pools of fragments in which (a) at least 90% derive from DNAse I hypersensitive sites; (b) 16% derive from core gene promoters; (c) are enriched for elements within 10 kb of gene transcription start sites; (d) are enriched for DNA elements conserved between mouse and human genome; and (e) are enriched for sequences with a considerably higher than expected density of transcription factor binding sites.

### Example 3

### Identification of Target Sequences of Estrogen Receptor (ER)

The human genome contains approximately 2,000 transcription factors that regulate every aspect of human development, adult ontogeny, and disease. Aberrant function of transcription factors causes disease: for example, breast cancer results from the aberrant function of the estrogen receptor (ER). Henderson et al. (2000) Carcinogenesis 21:427433. Although estrogen and the estrogen receptor are well established as causative agents of breast cancers, little is known about the regulatory network of breast epithelium response to ER. See, e.g., Sommer et al. (2001) Semin Cancer Biol 11:339-352; Sewacket al. (2001) Mol Cell Biol 21:1404-1415; Shang et al. (2000) Cell 103:843-852; and Ghosh et al. (2000) Cancer Res 60:6367-6375.

The primary obstacle to developing more effective therapeutic agents for breast cancer is thus the lack of information about the direct genomic targets of ER in the human genome. It is known that estrogen affects transcription of approximately 2,000 genes, but as little as 10 have been tentatively identified as direct targets. As a result of this information void, existing therapeutics that affect function of ER, e.g., tamoxifen, are only partly effective. If the direct targets of ER were known, then modulators of its function could be evaluated directly based on their effects on target genes most critical to disease onset and progression, but these direct targets remain largely unknown.

The following experiments are performed to identify direct target sequences of the ER transcription factor. Chromatin immunoprecipitation (ChIP) is conducted on human breast carcinoma line MCF-7 (ATCC Accession No. HTB-22) using an anti-ER antibody. See, for example, Kuo et al. (1999) Methods 19:425-433; O'Neill et al. (1999) Meth. Enzymology 274:189-197 and Orlando (2000) Trends Biochem. Sci. 25:99-104. Antibodies directed against the estrogen receptor are commercially available. Positive controls are obtained by analysis of known ER target genes including pS2 (Sewack et al. (2001) Mol Cell Biol 21:1404-1415); cathepsin W (Shang et al. (2000) Cell 103:843-852); PDZK1, and GREB1 (Ghosh et al. (2000) Cancer Res 60:6367-6375). Negative controls are obtained from MCF-7 cells cultured in the presence of estrogen and insulin because, under these culture conditions, ER does not bind to its target sites and relocates to the cytoplasm. Sommer et al. (2001) Semin Cancer Biol 11:339-352. Using these controls, only ChIP results that show at least 5-fold enrichment for core promoters of the positive control genes relative to the negative controls are selected for analysis on a regDNA chip.

To determine direct genomic targets of ER, the ChIP outputs from treated cells meeting these selection criteria are hybridized to a regDNA chip and the resulting pattern compared to the pattern of hybridization from ChIP performed on cells that were not treated with estrogen. Analysis is conduced essentially as described in Horak et al. (2002) Proc Nat'l Acad Sci USA 99:2924-2929; Ren et al. (2002) Geizes Dev 16:245-256; and Weinmarm et al. (2002) Genes Dev 16:235-244. The data is evaluated using three independent metrics: (1) increase of at least 2.5 fold of a signal for known ER targets over control targets (e.g., genes such as GAPDH, 13-actin); (2) positional analysis of identified DNA regulatory stretches bound by ER relative to genomic position of genes for which transcription is known to be affected by ER; and (3) target validation by manual analysis (e.g. using PCR with primers that amplify regulatory DNA identified by the regDNA chip to confimm binding of ER; see e.g., Martone et al (2003) supra).

### Example 4

### Analysis of Drug Effects

The following experiments are also conducted to determine the effect of estrogen and/or tamoxifen on gene activity in breast cancer cells.

A. Estrogen

Previously, more than 550 genes have been identified to be activated by least 3-fold, and approximately 450 have been shown to be repressed by at least about 2-fold, upon estrogen treatment of MCF-7 cells. Accordingly, to examine the effects of estrogen on regulatory sequences, MCF-7 cells are starved of estrogen and insulin for 7 days, and then half of the cells are treated with both hormones for 48 hrs. Regulatory DNA is prepared from both cell populations as described above and compared to the corresponding mRNA expression profile.

Duplicate batches of regulatory DNAs from estrogen treated and untreated cells are hybridized to regDNA chips. Expected results include at least a 2-fold decrease in regulatory DNA hybridization to the regDNA chip of 50% of those genes that are known to be repressed upon estrogen treatment. In addition, a positive correlation between gene activity and representation of its regulatory DNA in the regulatory DNA profile and low S.E.M. (<20% total signal) between biological duplicates is expected.

B. Estrogen and Tamoxifen

The nature of tissue-specific differences of tamoxifen action (which is anti-estrogenic in the breast and pro-estrogenic in the endometrium) is determined by comparing 4 datasets: (i) regDNA-wide distribution of ER in breast tissue following estrogen treatment; (ii) regDNA-wide distribution of ER in breast tissue following tamoxifen treatment; (iii) regDNA-wide distribution of ER in the endometrium following estrogen treatment; (iv) regDNA-wide distribution of ER in the endometrium following tamoxifen treatment.

Differences in the regDNA stretches occupied by ER in the breast are expected, depending on whether the tissue is treated with tamoxifen or estradiol. A large number of genes, however, will be bound by ER in breast tissue both in the presence of tamoxifen or estradiol—these will represent those ER targets most directly relevant to ER action in the breast. At the same time, it is expected that a large number of genes in the endometrium will be bound by ETR in the presence of both ligands. The critical step, therefore, will be to identify those genes that are bound by ER in the breast, but not in the endometrium, and vice versa. Furthermore, it will be critical to determine how ER distribution on those genes (assayed e.g. by ChIP on a regDNA chip) is affected by estrogen vs. tamoxifen treatment. Tissue-to-tissue and ligand-to ligand differences between these samples will illuminate genes directly relevant to the tissue-specific action by these ER ligands.

All references cited herein are hereby incorporated by reference in their entireties for all purposes. cl LIST OF REFERENCES

Birrell, G. W. et al. Transcriptional response of Saccharomyces cerevisiae to DNA-damaging agents does not identify the genes that protect against these agents. Proc Natl Acad Sci USA 99, 8778-83. (2002).

Bulger, M., Sawado, T., Schubeler, D. & Groudine, M. ChIPs of the beta-globin locus: unraveling gene regulation within an active domain. Curr Opin Genet Dev 12, 170-7. (2002).

Cox, J. M. & Papagallo, M. Contemporary and emergent pharmacological therapies for chronic pain: nonopiod analgesia. Expert Rev. Neurotherapeutics 1, 81-91 (2002).

Elgin, S. C. The formation and function of DNase I hypersensitive sites in the process of gene activation. J Biol Chem 263, 19259-62 (1988).

Galas, D. J. Sequence interpretation. Making sense of the sequence. Science 291, 1257-60. (2001)

Ghosh, M. G., Thompson, D. A. & Weigel, R. J. PDZK1 and GREB1 are estrogen-regulated genes expressed in hormone- responsive breast cancer. Cancer Res 60, 6367-75. (2000).

Giaever, G. et al. Functional profiling of the Saccharomyces cerevisiae genome. Nature 418, 387-91. (2002)

Gottgens, B. et al. Analysis of vertebrate SCL loci identifies consented enhancers. Nat Biotechnol 18, 181-6. (2000).

Gross, D. S. & Garrard, W. T. Nuclease hypersensitive sites in chromatin. Annu Rev Biochem 7 57, 159-97 (1988).

Hebbes, T. R., Clayton, A. L., Thorne, A. W. & Crane-Robinson, C. Core histone hyperacetylation co-maps with generalized DNase I sensitivity in the chicken b-globin chromosomal domain. EMBO J 13, 1823-30 (1994).

Henderson, B. E. & Feigelson, H. S. Hormonal carcinogenesis. Carcinogenesis 21, 427-33 (2000).

Horak, C. E. et al. GATA-1 binding sites mapped in the beta-globin locus by using mammalian chip-chip analysis. Proc Natl Acad Sci USA 99, 2924-2929. (2002).

Ibrahim, N. K. & Hortobagyi, G. N. The evolving role of specific estrogen receptor modulators (SERMs). Surg Oncol 8, 103-23 (1999).

Johnson, K. D. & Bresnick, E. H. Dissecting long-range transcriptional mechanisms by chromatin immunoprecipitation. Methods 26, 27-36. (2002).

Kozlova, T. & Thummel, C. S. Steroid Regulation of Postembryonic Development and Reproduction in Drosophila. Trends Endocrinol Metab 11, 276-280 (2000).

Nal, B., Mohr, E. & Ferrier, P. Location analysis of DNA-bound proteins at the whole-genome level: untangling transcriptional regulatory networks. Bioessays 23, 473-6. (2001)

Pennacchio, L. A. & Rubin, E. M. Genomic strategies to identify mammalian regulatory sequences. Nat Rev Genet 2, 100-9. (2001)

Pilpel, Y., Sudarsanam, P. & Church, G. M. Identifying regulatory networks by combinatorial analysis of promoter elements. Nat Genet 29, 153-9. (2001)

Ren, B. et al. E2F integrates cell cycle progression with DNA repair, replication, and G(2)/M checkpoints. Genes Dev 16, 245-56. (2002).

Ren, B. et al. Genome-wide location and function of DNA binding proteins. Science 290, 2306-9 (2000)

Sewack, G. F., Ellis, T. W. & Hansen, U. Binding of TATA Binding Protein to a Naturally Positioned Nucleosome Is Facilitated by Histone Acetylation. Mol Cell Biol 21, 1404-1415. (2001).

Shang, Y., Hu, X., DiRenzo, J., Lazar, M. A. & Brown, M. Cofactor dynamics and sufficiency in estrogen receptor-regulated transcription. Cell 103, 843-52 (2000).

Sieweke, M. Detection of transcription factor partners with a yeast one hybrid screen. Methods Mol Biol 130, 59-77 (2000).

Sommer, S. & Fuqua, S. A. Estrogen receptor and breast cancer. Semin Cancer Biol 11, 339-52. (2001).

Umov, F. D. A feel for the template: zinc finger protein transcription factors and chromatin. Biochem Cell Biol 80, 321-333 (2002).

Umov, F. D., Rebar, E. J., Reik, A. & Pandolfi, P. P. Designed transcription factors as structural, functional and therapeutic probes of chromatin in vivo: Fourth in review series on chromatin dynamics. EMBO Rep 3, 610-5. (2002).

Verreault, A. De novo nucleosome assembly: new pieces in an old puzzle. Genes Dev 14, 1430-8 (2000).

Weinnamn, A. S. & Famnham, P. J. Identification of unknown target genes of human transcription factors using chromatin immunoprecipitation. Methods 26, 3747. (2002).

Weinmann, A. S., Yan, P. S., Oberley, M. J., Huang, T. H. & Fariham, P. J. Isolating human transcription factor targets by coupling chromatin immunoprecipitation and CpG island microarray analysis. Genes Dev 16, 235-44. (2002).

Wingender, E. et al. The TRANSFAC system on gene expression regulation. Nucleic Acids Res 29, 281-3. (2001).

Wyrick, J. J. & Young, R. A. Deciphering gene expression regulatory networks. Curr Opin Genet Dev 12, 130-136 (2002)

What is claimed is:

1. A method for making an array, the method comprising:
(a) isolating a plurality of cellular polynucleotide sequences, whereby the sequences are isolated based on their accessibility in cellular chromatin; and
(b) attaching each of the isolated sequences to an address on a solid support.

2. An array comprising a plurality of accessible polynucleotide sequences, wherein:
(a) the sequences are isolated based on their accessibility in cellular chromatin; and
(b) each accessible sequence is located at a distinct address on a solid support.

3. The array of claim 2, wherein the accessible sequences are isolated from a plurality of different cell types from an organism.

4. The array of claim 2, wherein the accessible sequences are isolated from a single cell or tissue type from an organism.

5. The array of claim 2, wherein the accessible sequences are isolated according to the following procedure:
(a) isolating a first plurality of cellular polynucleotide sequences, whereby the sequences are isolated based on their accessibility in cellular chromatin from a first cell;
(b) isolating a second plurality of cellular polynucleotide sequences, whereby the sequences are isolated based on their accessibility in cellular chromatin from a second cell;
(c) obtaining sequences that are unique to either the first or second plurality of cellular polynucleotide sequences; and
(d) attaching each of the isolated sequences obtained in step (c) to an address on a solid support.

6. A method of identifying a target sequence bound by a DNA-binding protein, the method comprising the steps of:
(a) contacting at least one DNA-binding protein with an array according to claim 2, under conditions such that the protein binds to accessible sequences comprising a target sequence bound by the protein;
(b) removing unbound proteins; and
(c) identifying the accessible sequences bound by the protein, thereby identifing target sequences for the protein.

7. A method of identifying a transcription factor, the method comprising the steps of:
(a) preparing a preparation of proteins from a cell;
(b) contacting the isolated proteins with an array according to claim 2, under conditions such that transcription factors in the protein preparation bind to accessible sequences comprising a target sequence bound by a transcription factor;
(c) removing unbound proteins; and
(d) identifying the proteins bound to the array.

8. A method for obtaining a regulatory profile of accessible sequences in a cell, the method comprising:
(a) isolating a plurality of polynucleotide sequences from the cell, whereby the sequences are isolated based on their accessibility in cellular chromatin;
(b) optionally amplifying the sequences obtained in step (a);
(c) optionally labeling the sequences of step (a) or (b);
(d) contacting the sequences of step (a), (b) or (c) with an array according to claim 3; and
(e) identifying the accessible sequences bound on the array, thereby identifying sequences that are accessible in the cell.

9. A method for identifying functional binding sites for a DNA-binding protein in a cell, the method comprising:
(a) subjecting a cell to conditions under which DNA-binding proteins are crosslinked to their binding sites in cellular chromatin;
(b) shearing the crosslinked cellular chromatin of step (a);
(c) inmrunoprecipitating the sheared crosslinked chromatin of step (b) with an antibody which recognizes the DNA-binding protein;
(d) reversing the crosslinks in the immunoprecipitate of step (c);
(e) purifying the DNA from the irnmunoprecipitated material of step (d);
(f) optionally amplifying the DNA obtained in step (e);
(g) optionally labeling the DNA of step (e) or (f);
(h) contacting the DNA from step (e), (f) or (g) with an array according to claim 2; and
(i) identifying the accessible sequences bound on the array, thereby identifying functional binding sites for the DNA-binding protein in the cell.

10. A method of identifying a sequence in cellular chromatin, wherein the chromatin is covalently modified, the method comprising:
(a) providing a sample of cellular chromatin;
(b) optionally subjecting the chromatin of step (a) to conditions under which DNA-binding proteins are crosslinked to their binding sites in cellular chromatin;
(c) shearing the cellular chromatin of step (a) or (b);
(d) immunoprecipitating the sheared chromatin of step (c) with an antibody which recognizes a covalent chromatin modification;
(e) purifying the DNA from the immunoprecipitated material of step (d);
(f) optionally amplifying the DNA obtained in step (e);
(g) optionally labeling the DNA of step (e) or (f);
(h) contacting the DNA from step (e), (f) or (g) with an array according to claim 2; and
(i) identifying the accessible sequences bound on the array, thereby identifying sequences in cellular chromatin wherein the chromatin is covalently modified.

11. A method for characterizing the effects of a molecule on a cell, the method comprising:
(a) contacting the cell with the molecule;
(b) isolating a first plurality of polynucleotide sequences from the cell of step (a), whereby the sequences are isolated based on their accessibility in cellular chromatin;
(c) optionally amplifying the sequences obtained in step (b);
(d) optionally labeling the sequences of step (b) or (c);
(e) contacting the sequences of step (b), (c) or (d) with an array according to claim 2; and
(f) identifying the accessible sequences bound on the array, thereby identifying sequences that are accessible in the cell.

12. The method of claim 11, further comprising the steps of:
(g) providing cells that have not been contacted with the molecule;
(h) isolating a second plurality of polynucleotide sequences from the cell of step (g), whereby the sequences are isolated based on their accessibility in cellular chromatin;
(i) optionally amplifying the sequences obtained in step (h);
(j) obtaining sequences that are unique to either the first or second plurality of polynucleotide sequences;
(k) optionally amplifying the sequences obtained in step (j);
(l) optionally labeling the sequences of step (j) or (k);
(m) contacting the sequences of step (j), (k) or (1) with an array according to claim 2; and
(n) identifying the accessible sequences bound on the array, thereby identifying differences in accessible sequences between cells that have and have not been contacted with the molecule.

13. A method of identifying single nucleotide polymorphisms (SNPs) in regulatory sequences of an individual, the method comprising the steps of:
(a) preparing a library of regulatory DNA sequences from chromatin isolated from cells from the individual;
(b) optionally labeling the sequences of step (a);
(c) hybridizing the sequences of step (a) or (b) to an array according to claim 2 under stringent hybridization conditions, wherein the regulatory DNA sequences of the library hybridize to complementary accessible sequences on the array;
(d) removing regulatory DNA sequences of the library that are not bound to accessible sequences on the array; and
(e) identifying accessible sequences on the array that are not hybridized to regulatory DNA sequences of the library, wherein the unbound accessible sequences on the array suggest the presence of a SNP in regulatory sequences of the individual corresponding to the unbound accessible sequence.

14. A method for characterizing the effects of a stimulus on a cell, the method comprising:
(a) subjecting the cell to the stimulus;
(b) isolating a first plurality of polynucleotide sequences from the cell of step (a), whereby the sequences are isolated based on their accessibility in cellular chromatin;
(c) optionally amplifying the sequences obtained in step (b);
(d) optionally labeling the sequences of step (b) or (c);
(e) contacting the sequences of step (b), (c) or (d) with an array according to claim 2; and
(f) identifying the accessible sequences bound on the array, thereby identifying sequences that are accessible in the cell.

15. The method of claim 14, further comprising the steps of:
(g) providing cells that have not been subjected to the stimulus;
(h) isolating a second plurality of polynucleotide sequences from the cell of step (g), whereby the sequences are isolated based on their accessibility in cellular chromatin;
(i) optionally amplifying the sequences obtained in step (h);
(j) obtaining sequences that are unique to either the first or second plurality of polynucleotide sequences;
(k) optionally amplifying the sequences obtained in step (j);
(l) optionally labeling the sequences of step (j) or (k);
(m) contacting the sequences of step (j), (k) or (l) with an array according to claim 2; and
(n) identifying the accessible sequences bound on the array, thereby identifying differences in accessible sequences between cells that have and have not been subjected to the stimulus.
